(19) United States
(12) Patent Application Publication
(10) Pub. No.: US 2010/0273892 A1
(43) Pub. Date: Oct. 28, 2010

(54) FORMULATIONS OF TOCOTRIENOL QUINONES FOR THE TREATMENT OF OPHTHALMIC DISEASES

(76) Inventors: Guy M. MILLER, Monte Sereno, CA (US); William D. Shrader, Belmont, CA (US); Viktoria Kheifets, Mountain View, CA (US)

Correspondence Address: MORRISON & FOERSTER LLP, 755 PAGE MILL RD, PALO ALTO, CA, 94304-1018, US

(21) Appl. No.: 12/768,554

(22) Filed: Apr. 27, 2010

Related U.S. Application Data
(60) Provisional application No. 61/214,795, filed on Apr. 28, 2009.
(60) Provisional application No. 61/318,737, filed on Mar. 29, 2010.

(51) Int. Cl. A61K 31/12; A61P 27/02; A61P 25/28; A61P 27/06; A61P 25/16; A61P 7/10

(52) U.S. Cl. 514/688

(57) ABSTRACT

A formulation, comprising an ophthalmically effective amount of one or more tocotrienol quinones, particularly of alpha-tocotrienol quinone is disclosed. Use of a formulation comprising one or more tocotrienol quinones for the prevention, reduction, amelioration or treatment of ophthalmic disorders that are associated with a neurodegenerative or trauma disorder is also discussed. A method of treating or controlling the ocular symptoms associated with neurodegenerative diseases or trauma with a formulation comprising one or more tocotrienol quinones is also discussed. A method of treating or controlling the ocular symptoms associated with mitochondrial myopathies with a formulation comprising one or more tocotrienol quinones is also discussed.

### CROSS-REFERENCE TO RELATED APPLICATIONS

This application claims priority benefit of U.S. Provisional Patent Application Nos. 61/214,795, filed Apr. 28, 2009, and 61/318,737, filed Mar. 29, 2010. The entire contents of those applications are hereby incorporated by reference herein.

### DESCRIPTION

The present invention relates to a formulation comprising one or more tocotrienol quinones of Formula I or mixtures thereof as described herein, to prevent, reduce, ameliorate, or treat ophthalmic disorders, or to stop the progression of, or reverse, the loss of vision. The present invention relates to a formulation comprising one or more tocotrienol quinones of Formula I or mixtures thereof as described herein, to prevent, reduce, ameliorate, or treat ophthalmic disorders, or to stop the progression of, or reverse, the loss of vision associated with neurodegenerative diseases or trauma. The present invention relates to a formulation comprising one or more tocotrienol quinones of Formula I or mixtures thereof as described herein, to prevent, reduce, ameliorate, or treat ophthalmic disorders, or to stop the progression of, or reverse, the loss of vision associated with mitochondrial myopathies, not including Leber's Hereditary Optic Neuropathy (LHON) or Dominant Optic Atrophy (DOA).

### BACKGROUND OF THE INVENTION

Mitochondrial myopathies are a group of diseases caused by damage to the mitochondria—small, energy-producing structures that serve as the cells' “power plants.” Inherited changes in mitochondrial DNA can cause problems with growth, development, and function of the body's systems. These mutations disrupt the mitochondria's ability to efficiently generate energy for the cell and always affect worse the organs with highest energy need. Although the health consequences of inherited mitochondrial DNA mutations vary widely, some frequently observed features include abnormalities involving the eyes and vision, including but not limited to visual loss and blindness, ptosis, opthalmoplegia optic atrophy, acquired strabismus, and retinitis pigmentosa (Kosmoirsky, et al., Neurol. Clin. (1991) 9:147-61 and Biousse, V. et al., Curr. Opin. Neurol. (2003) 16 (1): 35-43).

Mitochondrial myopathies include but are not limited to Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency. This invention does not address the myopathies and associated ophthalmic disorders caused by Leber's Hereditary Optic Neuropathy (LHON), or by Dominant Optic Atrophy (DOA).

Many patients with mitochondrial myopathies including ataxia symptoms have eye movement abnormalities (especially slowed saccades, abnormal pursuit, and nystagmus), optic neuropathy (especially among patients with Friedrich's ataxia), and retinal degeneration (spinocerebellar ataxia); Gouw et al., Nature Genetics (1995) 10, 89-93.

Chronic Progressive External Opthalmoplegia (CPEO) is a disorder characterized by slowly progressive paralysis of the extraocular muscles. Patients usually experience bilateral, symmetrical, progressive ptosis, followed by opthalmoparesis months to years later. Ciliary and iris muscles are not involved. CPEO is the most frequent manifestation of mitochondrial myopathies. CPEO in association with mutations in mitochondrial DNA (mtDNA) may occur in the absence of any other clinical sign, but it is usually associated with skeletal muscle weakness.

Leigh's syndrome (also known as Leigh's disease or subacute necrotizing encephalomyelopathy) is one of many mitochondrial disorders. It is a progressive neurodegenerative disorder due to a wide variety of genetic mutations in mitochondrial DNA (mtDNA) or in nuclear DNA (gene SURF1 and some COX assembly factors). It is an inherited disorder that usually affects infants between the age of three months and two years, but, in rare cases, teenagers and adults as well. Some of the symptoms include loss of vision, and abnormal eye movements.

Typically symptoms present before the age of 2, with presentation in later childhood or adulthood being uncommon. Symptoms include psychomotor delay/regression with superimposed signs of basal ganglia and brain stem dysfunction: ataxia, opthalmoplegia, and dystonia.

Friedreich's ataxia (FRDA) is an autosomal recessive neurodegenerative and cardiodegenerative disorder caused by decreased levels of the protein frataxin. The disease causes the progressive loss of voluntary motor coordination (ataxia) and cardiac complications. Symptoms typically begin in childhood, and the disease progressively worsens as the patient grows older; patients eventually become wheelchair-bound due to motor disabilities. Patients with Friedreich's ataxia develop loss of visual acuity or changes in color vision. Most have jerky eye movements (nystagmus), but these movements by themselves do not necessarily interfere with vision.

Mitochondrial myopathy, Encephalopathy, Lactacidosis, and Stroke (MELAS) is a disease that can manifest itself in infants, children, or young adults. Ocular changes in MELAS syndrome have included reversible scotomata, opthalmoplegia, and pigmentary retinopathy.

Kearns-Sayre Syndrome (KSS) is characterized by a triad of features including: (1) typical onset in persons younger than age 20 years; (2) chronic, progressive, external opthalmoplegia; and (3) pigmentary degeneration of the retina. In addition, KSS may include cataracts.

Spinocerebellar ataxia (SCA), also called Machado-Joseph disease, is characterized by slowly progressive incoordination of gait and often associated with poor coordination of hands, speech, and eye movements. Nystagmus and macular degeneration are two characteristics of this disease. Gupta, S et al., (Journal of Neurological Sciences (2008) 264: 173-176) have disclosed the diagnosis of spinocerebellar ataxia with vision loss secondary to retinal pigmentary dystrophy.

Yet another devastating syndrome resulting from a respiratory chain disorder is Co-Enzyme Q10 (CoQ10) Deficiency, the symptoms of which include encephalomyopathy, mental retardation, exercise intolerance, ragged-red fibers, and recurrent myoglobin in the urine. CoQ10 Deficiency has also been associated with eye movement symptoms.

Yet other syndromes, named overlap syndromes, combine the clinical features of different typical mitochondrial syndromes. One such syndrome characterized by clinical features of both myoclonus epilepsy ragged-red fibers (MERRF) and Kearns-Sayre syndrome (KSS), and due to a mitochondrial DNA (mtDNA) mutation at nucleotide 3255 (G3255A) of the tRNA^{Leu(UUR)} gene has been described by Nishigaki, Y et al., Neuromuscular Disorders (2003) 13:334-340. This particular overlap syndrome manifests sensorineural deafness, atypical pigmentary retinopathy, myoclonus epilepsy, ptosis, opthalmoparesis, migraine headaches, hypothyroidism, and testosterone insufficiency.

Glaucoma is part of a group of diseases of the optic nerve involving loss of retinal ganglion cells in a characteristic pattern of optic neuropathy. Raised intraocular pressure is a significant risk factor for developing glaucoma (above 22 mmHg). One person may develop nerve damage at a relatively low pressure, while another person may have high eye pressure for years and yet never develop damage. Untreated glaucoma leads to permanent damage of the optic nerve and resultant visual field loss, which can progress to blindness.

Glaucoma can be divided roughly into two main categories, “open angle” or chronic glaucoma, and “closed angle” or acute glaucoma. Angle closure, acute glaucoma appears suddenly and often with painful side effects and so is usually diagnosed quickly, although damage and loss of vision can also occur very suddenly. Primary open-angle glaucoma (POAG) is a progressive disease leading to optic nerve damage and, ultimately, loss of vision. Glaucoma results in the neuronal degeneration of the retina and optic nerve head. Even with aggressive medical care and surgical treatment, the disease generally persists causing a gradual loss of retinal neurons, a decline of visual function, and ultimately blindness.

Diabetic retinopathy (DR) is a common complication of diabetes and a leading cause of legal blindness in working-age adults. The clinical hallmarks of DR include increased vascular permeability, leading to edema, and endothelial cell proliferation. Much of the research effort has been focused on vascular changes, but it is becoming apparent that other degenerative changes occur beyond the vascular cells of the retina. These include increased apoptosis, glial cell reactivity, microglial activation, and altered glutamate metabolism. When occurring together, these changes may be considered as neurodegenerative and could explain some of the functional deficits in vision that begin soon after the onset of diabetes.

Age-related macular degeneration (AMD) is a disease associated with aging that gradually destroys sharp, central vision. Central vision is needed for seeing objects clearly and for common daily tasks such as reading and driving. AMD affects the macula, the part of the eye that provides humans with the ability to see fine detail. AMD causes no pain. In some cases, AMD advances so slowly that people notice little change in their vision. In others, the disease progresses faster and may lead to a loss of vision or legal blindness in both eyes. AMD is a leading cause of vision loss in Americans 60 years of age and older. It occurs in two forms: wet and dry.

Other forms of macular degeneration (MD) sometimes covered under Juvenile Macular Degeneration (JMD) include Stargardt's disease, Best's vitelliform retinal dystrophy, Doyne's honeycomb retinal dystrophy, Malattia leventinese, Sorsby's fundus dystrophy, and Autosomal dominant hemorrhagic macular dystrophy. Stargardt's disease is the most common type of JMD. Symptoms typically develop in childhood or teen years. Symptoms include decline in visual acuity, drusen spots on the macula and scarring of the macula. Best's vitelliform retinal dystrophy, the second most common JMD, is usually a relatively mild form of macular degeneration. Its most distinctive symptom is an “egg yolk” large drusen spot on the macula at an early stage, which later breaks up into “scrambled egg” drusen.

Alzheimer's disease is a common progressive neurodegenerative disease that affects approximately 4 million people in the United States. In about one-third of Alzheimer's cases, there is a predominantly “visual” presentation in which symptoms of visual cortical dysfunction dominate. These patients usually present with vague complaints of poor vision, problems with way-finding, and problems reading.

Progressive Supranuclear Palsy (PSP) is a rare neurodegenerative disorder that combines an abnormality of voluntary eye movements with preserved vestibular ocular reflex movements, impaired postural reflexes with falling backwards, and Parkinsonism.

Parkinson Disease (PD) and other Parkinson-like diseases (called Parkinsonisms) frequently cause increasing vision problems as the illness progresses. As PD or a related disease progresses, many patients develop increasingly poor eyesight (functionally reduced visual acuity).

Patients with Amyotrophic Lateral Sclerosis (ALS) typically experience ocular abnormalities thought to be caused by dysfunction in the neural system that controls motor performance. Patients that have been on a ventilator for long periods may have a high frequency of ocular abnormalities, such as the inability to voluntary close the eyes, or complete ocular paralysis (opthalmoplegia). In some cases ALS patients suffer from double and blurred vision.

Some additional neurodegenerative diseases associated with optic neuropathy as described in Pelak, V. S. Opthalmol. Clin. N. Am. (2004), 17:311-320 include Chacot-Marie-Tooth Disease, Mucopolysaccharidoses, Adrenoleukodystrophy, Niemann-Pick disease, Krabbe's disease, Pelizaeus-Merzbacher disease, Subacute necrotizing encephalomyelopathy of Leigh, Progressive encephalopathy, edema, hypsarrhythmia and optic atrophy (PEHO).

Traumatic eye injuries occur from incidents such as being poked in the eye or hit on the head. Depending on the type of trauma, symptoms can include blurred vision, bulging eye, burning, double vision, dry eyes, floaters, light sensitivity and pain or discomfort of the eye or around the eye. Other occurrences that might occur include swelling, a pupil that is dilated or unresponsive to light, vision loss, limited eye or lid movement or ptosis (drooping eyelids). An estimated 10 to 13 percent of wounded Iraq war veterans have sustained direct, penetrating eye damage, typically as a result of modern weaponry that unleashes an explosive cascade of fragments. Some of these service members are suffering from injuries that stem from trauma in the brain affecting the visual neurological pathways.

Traumatic Optic Neuropathy (TON) refers to an acute injury of the optic nerve secondary to trauma. The optic nerve axons may be damaged either directly or indirectly and the visual loss may be partial or complete. An indirect injury to the optic nerve typically occurs from the transmission of forces to the optic canal from blunt head trauma. This is in contrast to direct TON, which results from an anatomical disruption of the optic nerve fibers from penetrating orbital trauma, bone fragments within the optic canal, or nerve sheath hematomas. Patients undergoing corneal transplant or stem cell transplant of eye cells may also undergo trauma.

Acute orbital compartment syndrome is a rare but treatable complication of increased pressure within the confined orbital space as a result of facial trauma. The condition presents with recognizable physical findings and progressive visual deficit.

The use of tocotrienol quinones of Formula I for the treatment of mitochondrial diseases has been described in co-owned patent publication US 2006/0281809, but this application does not describe formulations to prevent, reduce, ameliorate or treat ophthalmic disorders associated with neurodegenerative disorders or trauma.

Formula I

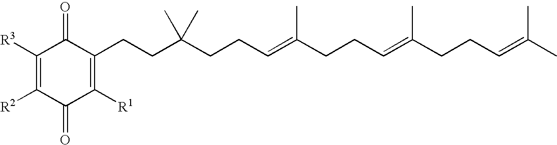

| | | | |
|---|---|---|---|
| Alpha-Tocotrienol quinone | R¹ = CH₃ | R² = CH₃ | R³ = CH₃ |
| Beta-Tocotrienol quinone | R₁ = CH₃ | R² = H | R³ = CH₃ |
| Gamma-Tocotrienol quinone | R₁ = H | R² = CH₃ | R³ = CH₃ |
| Delta-Tocotrienol quinone | R₁ = H | R² = H | R³ = CH₃ |

Tanito et al., Distribution of Tocopherols and Tocotrienols to Rat Ocular Tissues after Topical Ophthalmic Administration, Lipids, (2004), 39, No. 5:469-474, showed that the concentration of alpha-tocotrienol increased markedly in every tissue to which it was administered, and no significant increase was observed in the case of alpha-tocopherol. Tanito does not describe tocotrienol quinones.

The use of Vitamin E tocopheryl derivatives, not of tocotrienol or tocotrienol quinones, in ophthalmic compositions has been described in U.S. Pat. No. 5,886,030; however, these derivatives are used to increase the aqueous solubility of certain poorly soluble ophthalmic agents, not as the active compound in the amelioration, treatment or suppression of ophthalmic neurodegenerative diseases. It is however envisioned within the spirit of the invention that vitamin E tocopheryl derivatives might be included in the ocular formulations to provide additional comfort and non-irritability to said formulations.

The use of tocotrienols for the inhibition of the pathogen Chlamydia is described in patent publication US 2006/0241174. This publication claims but does not describe the mode of application of Vitamin E tocochromanol in the treatment of Chlamydia with eye drops. This publication does not describe any treatment with tocotrienol quinones.

### SUMMARY OF THE INVENTION

The invention relates to a formulation comprising an ophthalmically effective amount of one or more compounds of Formula I or mixtures thereof.

In one embodiment, the invention relates to a formulation comprising an ophthalmically effective amount of one or more compounds of Formula I or mixtures thereof additionally comprising a pharmaceutically or ophthalmically acceptable vehicle.

The invention relates to a formulation for preventing, reducing, ameliorating or treating ophthalmic disorders or for stopping the progression or reversing the loss of vision, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation is an oral formulation. In other embodiments, the formulation is a topical formulation.

In some embodiments, the invention relates to a formulation comprising an ophthalmically effective amount of alpha-tocotrienol quinone. In some embodiments, the alpha-tocotrienol quinone has a purity of 75% to 99%, or of about 75% to about 99%. In some embodiments, the formulation is an oral formulation. In other embodiments, the formulation is a topical formulation.

In another aspect, the invention relates to a formulation beneficial for a patient suffering from or at risk of ophthalmic disorders or vision loss, said formulation comprising an ophthalmically effective amount of one or more agents selected from the group consisting of alpha-tocotrienol quinone, beta-tocotrienol quinone, gamma-tocotrienol quinone, delta-tocotrienol quinone, or mixtures thereof; and an ophthalmically acceptable vehicle.

In another embodiment, the invention relates to a formulation comprising alpha-tocotrienol quinone to prevent, reduce, ameliorate or treat ophthalmic disorders in individuals in need of such treatment. In another embodiment, the invention relates to a formulation beneficial in a patient suffering from or at risk of ophthalmic disorders or vision loss, said formulation comprising an ophthalmically effective amount of alpha-tocotrienol quinone. In another embodiment, the invention relates to a formulation beneficial in a patient suffering from or at risk of ophthalmic disorders or vision loss, said formulation comprising an ophthalmically effective amount of alpha-tocotrienol quinone and an ophthalmically acceptable vehicle. In another embodiment, the invention relates to the use of a formulation comprising alpha-tocotrienol quinone having a purity of 75% to 99%, or of about 75% to about 99%, to prevent, reduce, ameliorate or treat ophthalmic disorders in individuals in need of such treatment.

In another embodiment, the invention relates to a formulation comprising beta-tocotrienol quinone to prevent, reduce, ameliorate or treat ophthalmic disorders in individuals in need of such treatment. In another embodiment, the invention relates to a formulation beneficial in a patient suffering from or at risk of ophthalmic disorders or vision loss, said formulation comprising an ophthalmically effective amount of beta-tocotrienol quinone. In another embodiment, the invention relates to a formulation beneficial in a patient suffering from or at risk of ophthalmic disorders or vision loss, said formulation comprising an ophthalmically effective amount of beta-tocotrienol quinone and an ophthalmically acceptable vehicle. In another embodiment, the invention relates to the use of a formulation comprising beta-tocotrienol quinone having a purity of 75% to 99%, or of about 75% to about 99%, to prevent, reduce, ameliorate or treat ophthalmic disorders in individuals in need of such treatment.

In another embodiment, the invention relates to a formulation comprising gamma-tocotrienol quinone to prevent, reduce, ameliorate or treat ophthalmic disorders in individuals in need of such treatment. In another embodiment, the invention relates to a formulation beneficial in a patient suffering from or at risk of ophthalmic disorders or vision loss, said formulation comprising an ophthalmically effective amount of gamma-tocotrienol quinone. In another embodiment, the invention relates to a formulation beneficial in a patient suffering from or at risk of ophthalmic disorders or vision loss, said formulation comprising an ophthalmically effective amount of gamma-tocotrienol quinone and an ophthalmically acceptable vehicle. In another embodiment, the invention relates to the use of a formulation comprising gamma-tocotrienol quinone having a purity of 75% to 99%, or of about 75% to about 99%, to prevent, reduce, ameliorate or treat ophthalmic disorders in individuals in need of such treatment.

In another embodiment, the invention relates to a formulation comprising delta-tocotrienol quinone to prevent, reduce, ameliorate or treat ophthalmic disorders in individuals in need of such treatment. In another embodiment, the invention relates to a formulation beneficial in a patient suffering from or at risk of ophthalmic disorders or vision loss, said formulation comprising an ophthalmically effective amount of delta-tocotrienol quinone. In another embodiment, the invention relates to a formulation beneficial in a patient suffering from or at risk of ophthalmic disorders or vision loss, said formulation comprising an ophthalmically effective amount of delta-tocotrienol quinone and an ophthalmically acceptable vehicle. In another embodiment, the invention relates to the use of a formulation comprising delta-tocotrienol quinone having a purity of 75% to 99%, or of about 75% to about 99%, to prevent, reduce, ameliorate or treat ophthalmic disorders in individuals in need of such treatment.

In another embodiment, the invention relates to a formulation for preventing, reducing, ameliorating or treating ophthalmic disorders associated with a neurodegenerative diseases or trauma, wherein the formulation comprises an ophthalmically effective amount of one or more agents of Formula I or mixtures thereof selected from the group consisting of alpha-tocotrienol quinone, beta-tocotrienol quinone, gamma-tocotrienol quinone, delta-tocotrienol quinone, or mixtures thereof. In some embodiments, the tocotrienol quinone of Formula I is alpha-tocotrienol quinone. In other embodiments, the formulation additionally comprises a pharmaceutically acceptable vehicle. In some embodiments, the formulation is an oral formulation. In other embodiments, the formulation is a topical formulation.

In another embodiment, the invention relates to a formulation comprising a tocotrienol quinone of Formula I or mixtures thereof, beneficial for the protection against, reduction, amelioration or treatment of an ophthalmic disorder associated with a disease selected from: inherited mitochondrial diseases, Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; Complex V deficiency; neurodegenerative diseases; Parkinson's disease; Alzheimer's disease; Amyotrophic Lateral Sclerosis (ALS); motor neuron diseases; other neurological diseases; Huntington's Disease; age-associated diseases; glaucoma and other diseases and disorders of the outer retina; macular degeneration, particularly age related macular degeneration or juvenile macular degeneration; retinal ischemia; acute retinopathies associated with trauma; post-surgical complications; traumatic optic neuropathy (TON), and the damage associated with laser therapy including photodynamic therapy (PDT), with surgical light induced iatrogenic retinopathy, and with corneal transplants and stem cell transplant of eye cells. In some embodiments, the disease is not LHON or DOA. In some embodiments, the tocotrienol quinone of Formula I is alpha-tocotrienol quinone. In other embodiments, the formulation additionally comprises a pharmaceutically acceptable vehicle.

In another embodiment, the invention relates to a formulation comprising a tocotrienol quinone of Formula I or mixtures thereof, and a pharmaceutically acceptable vehicle, beneficial for the protection against, reduction, amelioration or treatment of a mitochondrial myopathy selected from: inherited mitochondrial diseases, Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency. In another embodiment, the invention relates to a formulation comprising a tocotrienol quinone of Formula I or mixtures thereof and a pharmaceutically acceptable vehicle, beneficial for the protection against, reduction, amelioration or treatment of a mitochondrial myopathy resulting from an overlap syndrome characterized by clinical features of both myoclonus epilepsy ragged-red fibers (MERRF) and Kearns-Sayre syndrome (KSS), which is due to a mitochondrial DNA (mtDNA) mutation at nucleotide 3255 (G3255A) of the tRNA^{Leu(UUR)} gene.

In other embodiments, the invention relates to a formulation comprising a tocotrienol quinone of Formula I or mixtures thereof, and a pharmaceutically acceptable vehicle, beneficial for the protection against, reduction, amelioration or treatment of a mitochondrial myopathy that is not Leber's hereditary optic neuropathy or dominant optic neuropathy. In some embodiments, the tocotrienol quinone of Formula I is alpha-tocotrienol quinone. In other embodiments, the formulation additionally comprises a pharmaceutically acceptable vehicle.

In another embodiment, the invention relates to a formulation comprising a tocotrienol quinone of Formula I or mixtures thereof, beneficial for the protection against, reduction, amelioration or treatment of ophthalmic disorders associated with mitochondrial myopathies including inherited mitochondrial diseases; Chronic Progressive External Opthalmoplegia (CPEO); Spinocerebellar ataxia (SCA); also called Machado-Joseph disease, Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency. In some embodiments, the tocotrienol quinone of Formula I is alpha-tocotrienol quinone. In other embodiments, the formulation additionally comprises a pharmaceutically acceptable vehicle. In other embodiments, the formulation is an oral formulation. In other embodiments, the formulation is a topical formulation. In the foregoing embodiments, the formulation is not for the treatment of LHON or DOA.

In another embodiment, the invention relates to a formulation comprising a tocotrienol quinone of Formula I or mixtures thereof, beneficial for the protection against, reduction, amelioration or treatment of ophthalmic disorders associated with neurodegenerative disorders or trauma, including but not limited to Parkinson's disease; Alzheimer's disease; Amyotrophic Lateral Sclerosis (ALS); motor neuron diseases; other neurological diseases; Huntington's Disease; and age-associated diseases. In some embodiments, the tocotrienol quinone of Formula I is alpha-tocotrienol quinone. In other embodiments, the formulation additionally comprises a pharmaceutically acceptable vehicle. In other embodiments, the formulation is an oral formulation. In other embodiments, the formulation is a topical formulation.

In another embodiment, the invention relates to a formulation comprising a tocotrienol quinone of Formula I or mixtures thereof, beneficial for the protection against, reduction, amelioration or treatment of ophthalmic disorders associated with neurodegenerative disorders or trauma, including but not limited to glaucoma and other diseases and disorders of the outer retina; and macular degeneration, particularly age related macular degeneration. In some embodiments, the tocotrienol quinone of Formula I is alpha-tocotrienol quinone. In other embodiments, the formulation additionally comprises a pharmaceutically acceptable vehicle. In other embodiments, the formulation is an oral formulation. In other embodiments, the formulation is a topical formulation.

In another embodiment, the invention relates to a formulation comprising a tocotrienol quinone of Formula I or mixtures thereof, beneficial for the protection against, reduction, amelioration or treatment of ophthalmic disorders associated with trauma such as retinal ischemia, acute retinopathies associated with trauma, post-surgical complications, traumatic optic neuropathy (TON); and the damage associated with laser therapy including photodynamic therapy (PDT), with surgical light induced iatrogenic retinopathy, and with corneal transplants and stem cell transplant of eye cells. In some embodiments, the tocotrienol quinone of Formula I is alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In one embodiment, the invention relates to the use of a formulation comprising a tocotrienol quinone of Formula I or mixtures thereof, to prevent, reduce, ameliorate or treat ophthalmic disorders or to stop the progression of, or reverse, the loss of vision of a patient suffering from, or at risk of, mitochondrial myopathies excluding LHON and excluding DOA. In other embodiments, the mitochondrial myopathy is selected from the group consisting of inherited mitochondrial diseases; Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency with the proviso that the mitochondrial disease is not LHON or DOA. In other embodiments, the formulation additionally comprises a pharmaceutically acceptable vehicle. In some of the foregoing embodiments, the formulation is an oral formulation. In other embodiments, the formulation is a topical formulation.

In another embodiment, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of the mitochondrial myopathy selected from the group consisting of inherited mitochondrial diseases; Chronic Progressive External Opthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Syndrome; Kearns-Sayre Syndrome (KSS); overlap syndromes; and Friedreich's Ataxia (FRDA).

In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of an inherited mitochondrial disease. In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of the mitochondrial disorder, Chronic Progressive External Opthalmoplegia (CPEO). In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of Spinocerebellar ataxia (SCA), also called Machado-Joseph disease. In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of Friedreich's ataxia (FRDA). In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of Mitochondrial Myopathy, Encephalopathy, Lactacidosis, and Stroke (MELAS). In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of Kearns-Sayre Syndrome (KSS). In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of Leigh's syndrome. In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of Myoclonic Epilepsy with Ragged Red Fibers (MERRF). In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from or at risk of an overlap syndrome.

In another embodiment, the invention relates to the use of a formulation comprising a tocotrienol quinone of Formula I or mixtures thereof, to prevent, reduce, ameliorate or treat ophthalmic disorders or to stop the progression of, or reverse, the loss of vision of a patient suffering from or at risk of a neurodegenerative disorder associated with ophthalmic disorders or vision loss, wherein said neurodegenerative disorder is selected from the group consisting of glaucoma; diabetic retinopathy; macular degeneration including age-related macular degeneration and juvenile macular degeneration; Alzheimer's, Progressive Supranuclear palsy (PSP); Parkinson Disease (PD) and other Parkinson-like diseases (called Parkinsonisms); Amyotrophic lateral sclerosis (ALS); Chacot-Marie-Tooth Disease; Mucopolysaccharidoses, Adrenoleukodystrophy; Niemann-Pick disease; Krabbe's disease; Pelizaeus-Merzbacher disease; Subacute necrotizing encephalomyelopathy of Leigh; and Progressive encephalopathy, edema, hypsarrhythmia and optic atrophy (PEHO).

In another embodiment of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from Alzheimer's disease. In another embodiment of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from Progressive Supranuclear Palsy (PSP). In another embodiment of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from Parkinson Disease (PD) and other Parkinson-like diseases (called Parkinsonisms). In another embodiment of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from Amyotrophic Lateral Sclerosis (ALS). In some embodiments, the tocotrienol quinone of Formula I is alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from glaucoma. In other embodiments of the invention, the use of a formulation comprising a tocotrienol quinone of Formula I or mixtures thereof, is to prevent, reduce, ameliorate or treat ophthalmic disorders or to stop the progression of, or reverse, the loss of vision of a patient suffering from Primary Open-Angle Glaucoma (POAG). In some of the foregoing embodiments, the formulation is an oral formulation. In other embodiments, the formulation is a topical formulation.

In another embodiment of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from diabetic retinopathy (DR).

In another embodiment of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from macular degeneration (MD). In some embodiments of the invention, the use of a formulation comprising a tocotrienol quinone of Formula I or mixtures thereof is to prevent, reduce, ameliorate or treat ophthalmic disorders or to stop the progression of, or reverse, the loss of vision of a patient suffering from age-related macular degeneration (AMD). In other embodiments of the invention the use of a formulation comprising a tocotrienol quinone of Formula I or mixtures thereof, is to prevent, reduce, ameliorate or treat ophthalmic disorders or to stop the progression of, or reverse, the loss of vision of a patient suffering from juvenile macular degeneration (JMD).

In another embodiment, the invention relates to the use of a formulation comprising a tocotrienol quinone of Formula I or mixtures thereof to ameliorate or treat ophthalmic disorders or to stop the progression of, or reverse, the loss of vision of a patient suffering from traumatic eye injuries. In some embodiments, the traumatic injury is Traumatic Optic Neuropathy (TON). In other embodiments, the invention relates to the use of a tocotrienol quinone of Formula I or mixtures thereof for the amelioration or treatment of patients undergoing corneal transplants or stem cell transplant of eye cells.

In other embodiments, the invention relates to the use of a formulation comprising a tocotrienol quinone of Formula I or mixtures thereof for the amelioration or treatment of patients with acute retinopathies associated with trauma, post-surgical complications, traumatic optic neuropathy (TON); and the damage associated with laser therapy including photodynamic therapy (PDT), with surgical light induced iatrogenic retinopathy, and with corneal transplants and stem cell transplant of eye cells. In some embodiments, the tocotrienol quinone of Formula I is alpha-tocotrienol quinone. In other embodiments, the formulation additionally comprises a pharmaceutically or ophthalmically acceptable vehicle.

In another embodiment, including any of the foregoing embodiments, the use of a formulation comprising a tocotrienol quinone of Formula I or mixtures thereof, is by oral administration. In other embodiments, including any of the foregoing embodiments, the use of a formulation comprising a tocotrienol quinone of Formula I or mixtures thereof, is by topical administration.

In another embodiment, including any of the foregoing embodiments, the formulation comprising a tocotrienol quinone of Formula I or mixtures thereof are useful as prophylactics to prevent the occurrence of ophthalmic neurodegenerative diseases and loss of vision. In some embodiments, the tocotrienol quinone of Formula I is alpha-tocotrienol quinone. In other embodiments, the formulation additionally comprises a pharmaceutically acceptable vehicle.

In another embodiment, the invention relates to the use of a formulation comprising alpha-tocotrienol quinone to prevent, reduce, ameliorate or treat ophthalmic disorders or to stop the progression of, or reverse, the loss of vision of a patient suffering from, or at risk of, mitochondrial myopathies excluding LHON and excluding DOA. In other embodiments, the invention relates to the use of a formulation comprising alpha-tocotrienol quinone to prevent, reduce, ameliorate or treat ophthalmic disorders or to stop the progression of, or reverse, the loss of vision of a patient suffering from or at risk of the group consisting of inherited mitochondrial diseases; Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency with the proviso that the mitochondrial disease is not LHON or DOA. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to the use of a formulation comprising alpha-tocotrienol quinone to prevent, reduce, ameliorate or treat ophthalmic disorders or to stop the progression of, or reverse, the loss of vision of a patient suffering from or at risk of an inherited mitochondrial disease; Chronic Progressive External Opthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Syndrome; Kearns-Sayre Syndrome (KSS); overlap syndromes; and Friedreich's Ataxia (FRDA).

In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of an inherited mitochondrial disease. In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Chronic Progressive External Opthalmoplegia (CPEO). In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Spinocerebellar ataxia (SCA), also called Machado-Joseph disease. In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Friedreich's ataxia (FRDA). In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Mitochondrial Myopathy, Encephalopathy, Lactacidosis, and Stroke (MELAS). In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Kearns-Sayre Syndrome (KSS). In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Leigh's syndrome. In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of Myoclonic Epilepsy with Ragged Red Fibers (MERRF). In another embodiment of the invention, the invention relates to the use of a formulation comprising alpha-tocotrienol quinone to prevent, reduce, ameliorate or treat ophthalmic disorders or to stop the progression of, or reverse, the loss of vision of a patient suffering from or at risk of an overlap syndrome.

In another embodiment, the invention relates to the use of a formulation comprising alpha-tocotrienol quinone, to prevent, reduce, ameliorate or treat ophthalmic disorders or to stop the progression of, or reverse, the loss of vision of a patient suffering from or at risk of a neurodegenerative disorder associated with ophthalmic disorders or vision loss, wherein said neurodegenerative disorder is selected from the group consisting of glaucoma; diabetic retinopathy; macular degeneration including age-related macular degeneration and juvenile macular degeneration; Alzheimer's, Progressive Supranuclear palsy (PSP); Parkinson Disease (PD) and other Parkinson-like diseases (called Parkinsonisms); Amyotrophic lateral sclerosis (ALS); Chacot-Marie-Tooth Disease; Mucopolysaccharidoses, Adrenoleukodystrophy; Niemann-Pick disease; Krabbe's disease; Pelizaeus-Merzbacher disease; Subacute necrotizing encephalomyelopathy of Leigh; and Progressive encephalopathy, edema, hypsarrhythmia and optic atrophy (PEHO).

In another embodiment of the invention, the use of a formulation comprising alpha-tocotrienol quinone is to prevent, reduce, ameliorate or treat ophthalmic disorders or to stop the progression of, or reverse, the loss of vision of a patient suffering from Alzheimer's disease. In another embodiment of the invention, the use of a formulation comprising alpha-tocotrienol quinone is to prevent, reduce, ameliorate or treat ophthalmic disorders or to stop the progression of, or reverse, the loss of vision of a patient suffering from Progressive Supranuclear Palsy (PSP). In another embodiment of the invention, the use of a formulation comprising alpha-tocotrienol quinone is to prevent, reduce, ameliorate or treat ophthalmic disorders or to stop the progression of, or reverse, the loss of vision of a patient suffering from Parkinson Disease (PD) and other Parkinson-like diseases (called Parkinsonisms). In another embodiment of the invention, the use of a formulation comprising alpha-tocotrienol quinone of is to prevent, reduce, ameliorate or treat ophthalmic disorders or to stop the progression of, or reverse, the loss of vision of a patient suffering from Amyotrophic Lateral Sclerosis (ALS). In some embodiments, the alpha-tocotrienol quinone is alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment of the invention, the use of a formulation comprising alpha-tocotrienol quinone is to prevent, reduce, ameliorate or treat ophthalmic disorders or to stop the progression of, or reverse, the loss of vision of a patient suffering from glaucoma. In other embodiments of the invention, the use of a formulation comprising alpha-tocotrienol quinone is to prevent, reduce, ameliorate or treat ophthalmic disorders or to stop the progression of, or reverse, the loss of vision of a patient suffering from Primary Open-Angle Glaucoma (POAG).

In another embodiment of the invention, the use of a formulation comprising alpha-tocotrienol quinone is to prevent, reduce, ameliorate or treat ophthalmic disorders or to stop the progression of, or reverse, the loss of vision of a patient suffering from diabetic retinopathy (DR).

In another embodiment of the invention, the use of a formulation comprising alpha-tocotrienol quinone is to prevent, reduce, ameliorate or treat ophthalmic disorders or to stop the progression of, or reverse, the loss of vision of a patient suffering from macular degeneration (MD). In some embodiments of the invention, the use of a formulation comprising alpha-tocotrienol quinone is to prevent, reduce, ameliorate or treat ophthalmic disorders or to stop the progression of, or reverse, the loss of vision of a patient suffering from age-related macular degeneration (AMD). In other embodiments of the invention the use of a formulation comprising alpha-tocotrienol quinone is to prevent, reduce, ameliorate or treat ophthalmic disorders or to stop the progression of, or reverse, the loss of vision of a patient suffering from juvenile macular degeneration (JMD).

In another embodiment, the invention relates to the use of a formulation comprising alpha-tocotrienol quinone to ameliorate or treat ophthalmic disorders or to stop the progression of, or reverse, the loss of vision of a patient suffering from traumatic eye injuries. In some embodiments, the traumatic injury is Traumatic Optic Neuropathy (TON). In other embodiments, the invention relates to the use of alpha-tocotrienol quinone for the amelioration or treatment of patients undergoing corneal transplants or stem cell transplant of eye cells.

In other embodiments, the invention relates to the use of a formulation comprising alpha-tocotrienol quinone for the amelioration or treatment of patients with acute retinopathies associated with trauma, post-surgical complications, and the damage associated with laser therapy including photodynamic therapy (PDT), traumatic optic neuropathy (TON), surgical light induced iatrogenic retinopathy, corneal transplants and stem cell transplant of eye cells. In other embodiments, the formulation additionally comprises a pharmaceutically or ophthalmically acceptable vehicle.

In another embodiment, including any of the foregoing embodiments, the use of a formulation comprising alpha-tocotrienol quinone is by oral administration. In another embodiment, including any of the foregoing embodiments, the use of a formulation comprising alpha-tocotrienol quinone is by topical administration.

In another embodiment, including any of the foregoing embodiments, the formulations comprising alpha-tocotrienol quinone are useful as prophylactics to prevent the occurrence of ophthalmic neurodegenerative diseases and loss of vision. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with mitochondrial myopathies, excluding LHON and excluding DOA, comprising administering to a patient in need of such treatment a formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In another embodiment, the invention relates to a method of treating or controlling the ocular symptoms associated with Chronic Progressive External Opthalmoplegia (CPEO), comprising administering to a patient in need of such treatment a formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation comprises alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with Friedreich's ataxia (FRDA), comprising administering to a patient in need of such treatment a formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the ophthalmic formulation comprises alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with an overlap syndrome such as the overlap syndrome characterized by clinical features of both myoclonus epilepsy ragged-red fibers (MERRF) and Kearns-Sayre syndrome (KSS), which is due to a mitochondrial DNA (mtDNA) mutation at nucleotide 3255 (G3255A) of the tRNA^{Leu(UUR)} gene, comprising administering to a patient in need of such treatment a formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation comprises alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with neurodegenerative diseases or trauma, comprising administering to a patient in need of such treatment a formulation, wherein the formulation comprises a pharmaceutically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation comprises alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with glaucoma; diabetic retinopathy; macular degeneration including age-related macular degeneration and juvenile macular degeneration; Alzheimer's; Progressive Supranuclear palsy (PSP); Parkinson Disease (PD) and other Parkinson-like diseases (called Parkinsonisms); Amyotrophic lateral sclerosis (ALS); Chacot-Marie-Tooth Disease; Mucopolysaccharidoses; Adrenoleukodystrophy; Niemann-Pick disease; Krabbe's disease; Pelizaeus-Merzbacher disease; Subacute necrotizing encephalomyelopathy of Leigh; and Progressive encephalopathy, edema, hypsarrhythmia; and optic atrophy (PEHO) comprising administering to a patient in need of such treatment a formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation comprises alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with trauma, post-surgical complications, the damage associated with laser therapy including photodynamic therapy (PDT), traumatic optic neuropathy (TON), surgical light induced iatrogenic retinopathy, corneal transplants and stem cell transplant of eye cells, comprising administering to a patient in need of such treatment a formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation comprises alpha-tocotrienol quinone. In other embodiments, the formulation additionally comprises a pharmaceutically acceptable vehicle. In some of the foregoing embodiments, the formulation is an oral formulation. In other embodiments, the formulation is a topical formulation.

In another embodiment, the invention relates to a method of treating or controlling the ocular symptoms associated with mitochondrial myopathies, excluding LHON and excluding DOA, comprising administering to a patient in need of such treatment a formulation, wherein the formulation comprises an ophthalmically effective amount of alpha-tocotrienol quinone. In another embodiment, the invention relates to a method of treating or controlling the ocular symptoms associated with Chronic Progressive External Opthalmoplegia (CPEO), comprising administering to a patient in need of such treatment a formulation, wherein the formulation comprises an ophthalmically effective amount of alpha-tocotrienol quinone. In other embodiments, the formulation additionally comprises a pharmaceutically acceptable vehicle.

In another embodiment, the invention relates to a method of treating or controlling the ocular symptoms associated with Friedreich's ataxia (FRDA), comprising administering to a patient in need of such treatment a formulation, wherein the formulation comprises an ophthalmically effective amount of alpha-tocotrienol quinone. In other embodiments, the formulation additionally comprises a pharmaceutically acceptable vehicle.

In another embodiment, the invention relates to a method of treating or controlling the ocular symptoms associated with an overlap syndrome such as the overlap syndrome characterized by clinical features of both myoclonus epilepsy ragged-red fibers (MERRF) and Kearns-Sayre syndrome (KSS), which is due to a mitochondrial DNA (mtDNA) mutation at nucleotide 3255 (G3255A) of the tRNA^{Leu(UUR)} gene, comprising administering to a patient in need of such treatment a formulation, wherein the formulation comprises an ophthalmically effective amount of alpha-tocotrienol quinone. In other embodiments, the formulation additionally comprises a pharmaceutically acceptable vehicle.

In another embodiment, the invention relates to a method of treating or controlling the ocular symptoms associated with neurodegenerative diseases or trauma, comprising administering to a patient in need of such treatment a formulation, wherein the formulation comprises a pharmaceutically effective amount of alpha-tocotrienol quinone. In some embodiments, the formulation comprises alpha-tocotrienol quinone. In other embodiments, the formulation additionally comprises a pharmaceutically acceptable vehicle.

In another embodiment, the invention relates to a method of treating or controlling the ocular symptoms associated with glaucoma; diabetic retinopathy; macular degeneration including age-related macular degeneration and juvenile macular degeneration; Alzheimer's; Progressive Supranuclear palsy (PSP); Parkinson Disease (PD) and other Parkinson-like diseases (called Parkinsonisms); Amyotrophic lateral sclerosis (ALS); Chacot-Marie-Tooth Disease; Mucopolysaccharidoses; Adrenoleukodystrophy; Niemann-Pick disease; Krabbe's disease; Pelizaeus-Merzbacher disease; Subacute necrotizing encephalomyelopathy of Leigh; and Progressive encephalopathy, edema, hypsarrhythmia; and optic atrophy (PEHO) comprising administering to a patient in need of such treatment a formulation, wherein the formulation comprises an ophthalmically effective amount of alpha-tocotrienol quinone. In other embodiments, the formulation additionally comprises a pharmaceutically acceptable vehicle.

In another embodiment, the invention relates to a method of treating or controlling the ocular symptoms associated with trauma, post-surgical complications, the damage associated with laser therapy including photodynamic therapy (PDT), traumatic optic neuropathy (TON), surgical light induced iatrogenic retinopathy, corneal transplants and stem cell transplant of eye cells, comprising administering to a patient in need of such treatment a formulation, wherein the formulation comprises an ophthalmically effective amount of alpha-tocotrienol quinone. In other embodiments, the formulation additionally comprises a pharmaceutically acceptable vehicle.

The invention also relates to a topical, periocular, or intraocular ophthalmic formulation for preventing, reducing, ameliorating or treating ophthalmic disorders; or for stopping the progression or reversing the loss of vision, wherein said ophthalmic formulation comprises an ophthalmically effective amount of alpha-tocotrienol quinone.

In some embodiments, the invention relates to a topical, periocular, or intraocular ophthalmic formulation comprising an ophthalmically effective amount of alpha-tocotrienol quinone. In some embodiments, the alpha-tocotrienol quinone has a purity of 75% to 99%, or of about 75% to about 99%.

In some embodiments, the ophthalmic formulations of the present invention are administered locally in eye drops. In other embodiments, the ophthalmic formulations of the present invention are administered as an irrigating solution. In other embodiments, the ophthalmic formulations of the present invention are administered periocularly. In other embodiments, the ophthalmic formulations of the present invention are administered intraocularly.

In another aspect, the invention relates to a topical, periocular, or intraocular ophthalmic formulation beneficial for neuroprotection in a patient suffering from or at risk of ophthalmic disorders or vision loss, said formulation comprising an ophthalmically effective amount of one or more agents selected from the group consisting of alpha-tocotrienol quinone, beta-tocotrienol quinone, gamma-tocotrienol quinone, delta-tocotrienol quinone, or mixtures thereof; and an ophthalmically acceptable vehicle.

In another embodiment, the invention relates to a topical, periocular, or intraocular ophthalmic formulation beneficial for neuroprotection in a patient suffering from or at risk of ophthalmic disorders or vision loss, said formulation comprising an ophthalmically effective amount of alpha-tocotrienol quinone. In another embodiment, the invention relates to a topical, periocular, or intraocular ophthalmic formulation beneficial for neuroprotection in a patient suffering from or at risk of ophthalmic disorders or vision loss, said formulation comprising an ophthalmically effective amount of alpha-tocotrienol quinone and an ophthalmically acceptable vehicle. In another embodiment, the invention relates to the topical, periocular, or intraocular use of a formulation comprising alpha-tocotrienol quinone having a purity of 75% to 99%, or of about 75% to about 99%, to prevent, reduce, ameliorate or treat ophthalmic disorders in individuals in need of such treatment. In another embodiment, the invention relates to the topical, periocular, or intraocular use of a formulation comprising beta-tocotrienol quinone to prevent, reduce, ameliorate or treat ophthalmic disorders in individuals in need of such treatment. In another embodiment, the invention relates to the topical, periocular, or intraocular use of a formulation comprising gamma-tocotrienol quinone to prevent, reduce, ameliorate or treat ophthalmic disorders in individuals in need of such treatment. In another embodiment, the invention relates to the topical, periocular, or intraocular use of a formulation comprising delta-tocotrienol quinone to prevent, reduce, ameliorate or treat ophthalmic disorders in individuals in need of such treatment.

In another embodiment, the invention relates to a topical, periocular, or intraocular ophthalmic formulation for preventing, reducing, ameliorating or treating ophthalmic disorders associated with a neurodegenerative diseases or trauma, wherein said ophthalmic formulation comprises an ophthalmically effective amount of one or more agents of Formula I or mixtures thereof selected from the group consisting of alpha-tocotrienol quinone, beta-tocotrienol quinone, gamma-tocotrienol quinone, delta-tocotrienol quinone, or mixtures thereof. In some embodiments, the tocotrienol quinone of Formula I is alpha-tocotrienol quinone. In other embodiments, the formulation additionally comprises an ophthalmically acceptable vehicle.

In another embodiment, the invention relates to a topical, periocular, or intraocular ophthalmic formulation comprising a tocotrienol quinone of Formula I or mixtures thereof, beneficial for the protection against, reduction, amelioration or treatment of an ophthalmic disorder associated with a disease selected from: inherited mitochondrial diseases; Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; Complex V deficiency; neurodegenerative diseases; Parkinson's disease; Alzheimer's disease; Amyotrophic Lateral Sclerosis (ALS); motor neuron diseases; other neurological diseases; Huntington's Disease; age-associated diseases; glaucoma and other diseases and disorders of the outer retina; macular degeneration, particularly age related macular degeneration or juvenile macular degeneration; retinal ischemia; acute retinopathies associated with trauma; post-surgical complications; traumatic optic neuropathy (TON); and the damage associated with laser therapy including photodynamic therapy (PDT); with surgical light induced iatrogenic retinopathy; and with corneal transplants and stem cell transplant of eye cells. In some embodiments, the tocotrienol quinone of Formula I is alpha-tocotrienol quinone. In other embodiments, the formulation additionally comprises an ophthalmically acceptable vehicle.

In another embodiment, the invention relates to a topical, periocular, or intraocular ophthalmic formulation comprising a tocotrienol quinone of Formula I or mixtures thereof, and an ophthalmically acceptable vehicle, beneficial for the protection against, reduction, amelioration or treatment of a mitochondrial myopathy selected from: inherited mitochondrial diseases; Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency. In other embodiments, the invention relates to a topical, periocular, or intraocular ophthalmic formulation comprising a tocotrienol quinone of Formula I or mixtures thereof, and an ophthalmically acceptable vehicle, beneficial for the protection against, reduction, amelioration or treatment of a mitochondrial myopathy that is not Leber's hereditary optic neuropathy or dominant optic neuropathy. In some embodiments, the tocotrienol quinone of Formula I is alpha-tocotrienol quinone. In other embodiments, the formulation additionally comprises an ophthalmically acceptable vehicle.

In another embodiment, the invention relates to a topical, periocular, or intraocular ophthalmic formulation beneficial for the protection against, reduction, amelioration or treatment of Chronic Progressive External Opthalmoplegia (CPEO), said formulation comprising an ophthalmically effective amount of a tocotrienol quinone of Formula I or mixtures thereof. In another embodiment, the invention relates to a topical, periocular, or intraocular ophthalmic formulation beneficial for the protection against ocular symptoms from Chronic Progressive External Opthalmoplegia (CPEO), said formulation comprising an ophthalmically effective amount of a tocotrienol quinone of Formula I or mixtures thereof. In another embodiment, the invention relates to a topical ophthalmic formulation beneficial for the reduction of ocular symptoms from Chronic Progressive External Opthalmoplegia (CPEO), said formulation comprising an ophthalmically effective amount of a tocotrienol quinone of Formula I or mixtures thereof. In another embodiment, the invention relates to a topical, periocular, or intraocular ophthalmic formulation beneficial for the amelioration of ocular symptoms from Chronic Progressive External Opthalmoplegia (CPEO), said formulation comprising an ophthalmically effective amount of a tocotrienol quinone of Formula I or mixtures thereof. In another embodiment, the invention relates to a topical, periocular, or intraocular ophthalmic formulation beneficial for the treatment of ocular symptoms from Chronic Progressive External Opthalmoplegia (CPEO), said formulation comprising an ophthalmically effective amount of a tocotrienol quinone of Formula I or mixtures thereof. In some embodiments, including any of the foregoing embodiments, the tocotrienol quinone of Formula I is alpha-tocotrienol quinone. In some embodiments, including any of the foregoing embodiments, the formulation additionally comprises an ophthalmically acceptable vehicle.

In another embodiment, the invention relates to a topical, periocular, or intraocular ophthalmic formulation beneficial for the protection against, reduction, amelioration or treatment of Chronic Progressive External Opthalmoplegia (CPEO), said formulation comprising an ophthalmically effective amount of alpha-tocotrienol quinone. In another embodiment, the invention relates to a topical, periocular, or intraocular ophthalmic formulation beneficial for the protection against ocular symptoms from Chronic Progressive External Opthalmoplegia (CPEO), said formulation comprising an ophthalmically effective amount of alpha-tocotrienol quinone. In another embodiment, the invention relates to a topical, periocular, or intraocular ophthalmic formulation beneficial for the reduction of ocular symptoms from Chronic Progressive External Opthalmoplegia (CPEO), said formulation comprising an ophthalmically effective amount of alpha-tocotrienol quinone. In another embodiment, the invention relates to a topical ophthalmic formulation beneficial for the amelioration of ocular symptoms from Chronic Progressive External Opthalmoplegia (CPEO), said formulation comprising an ophthalmically effective amount of alpha-tocotrienol quinone. In another embodiment, the invention relates to a topical, periocular, or intraocular ophthalmic formulation beneficial for the treatment of ocular symptoms from Chronic Progressive External Opthalmoplegia (CPEO), said formulation comprising an ophthalmically effective amount of alpha-tocotrienol quinone. In some embodiments, including any of the foregoing embodiments, the formulation additionally comprises an ophthalmically acceptable vehicle.

In another embodiment, the invention relates to a topical, periocular, or intraocular ophthalmic formulation comprising a tocotrienol quinone of Formula I or mixtures thereof, beneficial for the protection against, reduction, amelioration or treatment of ophthalmic disorders associated with mitochondrial myopathies including inherited mitochondrial diseases; Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF), Kearns-Sayre Syndrome (KSS); overlap syndromes; Co-Enzyme Q10 (CoQ10) Deficiency; Complex I deficiency; Complex II deficiency; Complex III deficiency; Complex IV deficiency, and Complex V deficiency. In some embodiments, the tocotrienol quinone of Formula I is alpha-tocotrienol quinone. In other embodiments, the formulation additionally comprises an ophthalmically acceptable vehicle.

In another embodiment, the invention relates to a topical, periocular, or intraocular ophthalmic formulation comprising a tocotrienol quinone of Formula I or mixtures thereof, beneficial for the protection against, reduction, amelioration or treatment of ophthalmic disorders associated with neurodegenerative disorders or trauma, including but not limited to Parkinson's disease; Alzheimer's disease; Amyotrophic Lateral Sclerosis (ALS); motor neuron diseases; other neurological diseases; Huntington's Disease; age-associated diseases; glaucoma and other diseases and disorders of the outer retina, macular degeneration, particularly age related macular degeneration; retinal ischemia; acute retinopathies associated with trauma; post-surgical complications; traumatic optic neuropathy (TON); and the damage associated with laser therapy including photodynamic therapy (PDT), with surgical light induced iatrogenic retinopathy, and with corneal transplants and stem cell transplant of eye cells. In some embodiments, the tocotrienol quinone of Formula I is alpha-tocotrienol quinone. In other embodiments, the formulation additionally comprises an ophthalmically acceptable vehicle.

In another embodiment, the invention relates to a topical, periocular, or intraocular ophthalmic formulation comprising a tocotrienol quinone of Formula I or mixtures thereof, beneficial for the protection against, reduction, amelioration or treatment of ophthalmic disorders associated with trauma such as retinal ischemia, acute retinopathies associated with trauma; post-surgical complications; traumatic optic neuropathy (TON); and the damage associated with laser therapy including photodynamic therapy (PDT), with surgical light induced iatrogenic retinopathy, and with corneal transplants and stem cell transplant of eye cells. In some embodiments, the tocotrienol quinone of Formula I is alpha-tocotrienol quinone. In other embodiments, the formulation additionally comprises an ophthalmically acceptable vehicle.

In another aspect, the invention relates to the use of a topical, periocular, or intraocular ophthalmic formulation comprising a tocotrienol quinone of Formula I or mixtures thereof, to prevent, reduce, ameliorate or treat ophthalmic disorders in individuals in need of such treatment. In one embodiment, the invention relates to the use of a topical, periocular, or intraocular ophthalmic formulation comprising alpha-tocotrienol quinone, to prevent, reduce, ameliorate or treat ophthalmic disorders in individuals in need of such treatment. In another embodiment, the invention relates to the topical, periocular, or intraocular use of a formulation comprising alpha-tocotrienol quinone having a purity of 75% to 99%, or of about 75% to about 99%, to prevent, reduce, ameliorate or treat ophthalmic disorders in individuals in need of such treatment.

In one embodiment, the invention relates to use of a topical, periocular, or intraocular ophthalmic formulation comprising beta-tocotrienol quinone to prevent, reduce, ameliorate or treat ophthalmic disorders in individuals in need of such treatment. In one embodiment, the invention relates to the use of a topical, periocular, or intraocular ophthalmic formulation comprising gamma-tocotrienol quinone to prevent, reduce, ameliorate or treat ophthalmic disorders in individuals in need of such treatment. In one embodiment, the invention relates to the use of a topical, periocular, or intraocular ophthalmic formulation comprising delta-tocotrienol quinone to prevent, reduce, ameliorate or treat ophthalmic disorders in individuals in need of such treatment.

In one embodiment, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from or at risk of mitochondrial myopathies excluding LHON and excluding DOA. In other embodiments, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from a mitochondrial myopathy selected from the group consisting of an inherited mitochondrial disease; Chronic Progressive External Opthalmoplegia (CPEO); Spinocerebellar ataxia (SCA), also called Machado-Joseph disease; Leigh's Syndrome; Friedreich's ataxia (FRDA); Mitochondrial myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency. In some embodiments, the tocotrienol quinone of Formula I is alpha-tocotrienol quinone. In other embodiments, the formulation additionally comprises a pharmaceutically acceptable vehicle. In other embodiments, the formulation additionally comprises an ophthalmically acceptable vehicle.

In another embodiment, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from mitochondrial myopathy associated with ophthalmic disorders or vision loss selected from the group consisting of inherited mitochondrial diseases; Chronic Progressive External Opthalmoplegia (CPEO); Spinocerebellar ataxia (SCA), also called Machado-Joseph disease; Myoclonic Epilepsy with Ragged Red Fibers (MERRF); Mitochondrial Myopathy, Encephalopathy, Lactacidosis, Stroke (MELAS); Leigh's Disease; Kearns-Sayre Syndrome (KSS); Friedreich's Ataxia (FRDA); and overlap syndromes.

In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from an inherited mitochondrial disease. In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Chronic Progressive External Opthalmoplegia (CPEO). In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Spinocerebellar ataxia (SCA), also called Machado-Joseph disease. In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Friedreich's ataxia (FRDA). In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from the mitochondrial disorder Mitochondrial Myopathy, Encephalopathy, Lactacidosis, and Stroke (MELAS). In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from the mitochondrial disorder Kearns-Sayre Syndrome (KSS). In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from the mitochondrial disorder Leigh's syndrome. In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Myoclonic Epilepsy with Ragged Red Fibers (MERRF). In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from an overlap syndrome. In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from the mitochondrial disorder characterized by clinical features of both myoclonus epilepsy ragged-red fibers (MERRF) and Kearns-Sayre syndrome (KSS), which is due to a mitochondrial DNA (mtDNA) mutation at nucleotide 3255 (G3255A) of the tRNA^{Leu(UUR)} gene.

In another embodiment, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from or at risk of a neurodegenerative disorder associated with ophthalmic disorders or vision loss, wherein said neurodegenerative disorder is selected from the group consisting of glaucoma; diabetic retinopathy; macular degeneration including age-related macular degeneration and juvenile macular degeneration; Alzheimer's, Progressive Supranuclear palsy (PSP); Parkinson Disease (PD) and other Parkinson-like diseases (called Parkinsonisms); Amyotrophic lateral sclerosis (ALS), Chacot-Marie-Tooth Disease; Mucopolysaccharidoses; Adrenoleukodystrophy; Niemann-Pick disease; Krabbe's disease; Pelizaeus-Merzbacher disease; Subacute necrotizing encephalomyelopathy of Leigh; and Progressive encephalopathy, edema, hypsarrhythmia and optic atrophy (PEHO).

In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Alzheimer's disease. In another embodiment of the invention the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Progressive Supranuclear Palsy (PSP). In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Parkinson Disease (PD) and other Parkinson-like diseases (called Parkinsonisms). In another embodiment of the invention the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Amyotrophic Lateral Sclerosis (ALS). In some embodiments, the tocotrienol quinone of Formula I is alpha-tocotrienol quinone. In other embodiments, the formulation additionally comprises a pharmaceutically acceptable vehicle. In other embodiments, the formulation additionally comprises an ophthalmically acceptable vehicle.

In another embodiment, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from glaucoma. In other embodiments of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Primary Open-Angle Glaucoma (POAG).

In another embodiment, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from diabetic retinopathy (DR).

In another embodiment of the invention, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from macular degeneration (MD). In some embodiments, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from age-related macular degeneration (AMD). In other embodiments, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from juvenile macular degeneration (JMD).

In another embodiment, the invention relates to the use of a topical, periocular, or intraocular ophthalmic formulation comprising a tocotrienol quinone of Formula I or mixtures thereof to ameliorate or treat ophthalmic disorders or to stop the progression of, or reverse, the loss of vision of a patient suffering from traumatic eye injuries. In some embodiments, the invention relates to the use of a topical, periocular, or intraocular ophthalmic formulation comprising a tocotrienol quinone of Formula I or mixtures thereof, to prevent, reduce, ameliorate or treat ophthalmic disorders or to stop the progression of, or reverse, the loss of vision of a patient suffering from Traumatic Optic Neuropathy (TON). In other embodiments, the invention relates to the use of a topical, periocular, or intraocular ophthalmic formulation comprising a tocotrienol quinone of Formula I or mixtures thereof, for the amelioration or treatment of patients undergoing corneal transplants or stem cell transplant of eye cells.

In other embodiments, the invention relates to the use of a topical, periocular, or intraocular ophthalmic formulation comprising a tocotrienol quinone of Formula I or mixtures thereof for the amelioration or treatment of patients with acute retinopathies associated with trauma, post-surgical complications, traumatic optic neuropathy (TON), and the damage associated with laser therapy including photodynamic therapy (PDT), with surgical light induced iatrogenic retinopathy, and with corneal transplants and stem cell transplant of eye cells. In some embodiments, the tocotrienol quinone of Formula I is alpha-tocotrienol quinone. In other embodiments, the formulation additionally comprises a pharmaceutically acceptable vehicle. In other embodiments, the formulation additionally comprises an ophthalmically acceptable vehicle.

In another embodiment, including any of the foregoing embodiments, the use of a topical, periocular, or intraocular ophthalmic formulation comprising a tocotrienol quinone of Formula I or mixtures thereof, is by topical administration. In another embodiment, including any of the foregoing embodiments, the use of a formulation comprising a tocotrienol quinone of Formula I or mixtures thereof, is by periocular administration. In another embodiment, including any of the foregoing embodiments, the use of a formulation comprising a tocotrienol quinone of Formula I or mixtures thereof, is by intraocular administration. In some embodiments, the tocotrienol quinone of Formula I is alpha-tocotrienol quinone. In other embodiments, the formulation additionally comprises a pharmaceutically acceptable vehicle. In other embodiments, the formulation additionally comprises an ophthalmically acceptable vehicle.

In another embodiment, including any of the foregoing embodiments, the formulation comprising a tocotrienol quinone of Formula I or mixtures thereof are useful as prophylactics to prevent the occurrence of ophthalmic neurodegenerative diseases and loss of vision. In some embodiments, the tocotrienol quinone of Formula I is alpha-tocotrienol quinone. In other embodiments, the formulation additionally comprises a pharmaceutically acceptable vehicle. In other embodiments, the formulation additionally comprises an ophthalmically acceptable vehicle.

In another embodiment, the invention relates to a method of treating or controlling the ocular symptoms associated with mitochondrial myopathies, excluding LHON and excluding DOA, comprising administering to a patient in need of such treatment a topical, periocular, or intraocular formulation, wherein said formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In another embodiment, the invention relates to a method of treating or controlling the ocular symptoms associated with Chronic Progressive External Opthalmoplegia (CPEO), comprising administering to a patient in need of such treatment a topical, periocular, or intraocular formulation, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the formulation comprises alpha-tocotrienol quinone. In other embodiments, the formulation additionally comprises a pharmaceutically acceptable vehicle. In other embodiments, the formulation additionally comprises an ophthalmically acceptable vehicle.

In another embodiment, the invention relates to a method of treating or controlling the ocular symptoms associated with Friedreich's ataxia (FRDA), comprising administering to a patient in need of such treatment a topical, periocular, or intraocular ophthalmic formulation, wherein said formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the topical, periocular, or intraocular ophthalmic formulation comprises alpha-tocotrienol quinone. In other embodiments, the topical, periocular, or intraocular formulation additionally comprises a pharmaceutically acceptable vehicle. In other embodiments, the topical, periocular, or intraocular formulation additionally comprises an ophthalmically acceptable vehicle.

In another embodiment, the invention relates to a method of treating or controlling the ocular symptoms associated with an overlap syndrome such as the overlap syndrome characterized by clinical features of both myoclonus epilepsy ragged-red fibers (MERRF) and Kearns-Sayre syndrome (KSS), which is due to a mitochondrial DNA (mtDNA) mutation at nucleotide 3255 (G3255A) of the tRNA^{Leu(UUR)} gene, comprising administering to a patient in need of such treatment a topical, periocular, or intraocular ophthalmic formulation, wherein said formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the topical ophthalmic formulation comprises alpha-tocotrienol quinone. In other embodiments, the topical, periocular, or intraocular formulation additionally comprises a pharmaceutically acceptable vehicle. In other embodiments, the topical, periocular, or intraocular formulation additionally comprises an ophthalmically acceptable vehicle.

In another embodiment, the invention relates to a method of treating or controlling the ocular symptoms associated with neurodegenerative diseases or trauma, comprising administering to a patient in need of such treatment a topical, periocular, or intraocular formulation, wherein said formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the topical, periocular, or intraocular ophthalmic formulation comprises alpha-tocotrienol quinone. In other embodiments, the topical, periocular, or intraocular formulation additionally comprises a pharmaceutically acceptable vehicle. In other embodiments, the topical, periocular, or intraocular formulation additionally comprises an ophthalmically acceptable vehicle.

In another embodiment, the invention relates to a method of treating or controlling the ocular symptoms associated with glaucoma; diabetic retinopathy; macular degeneration including age-related macular degeneration and juvenile macular degeneration; Alzheimer's; Progressive Supranuclear palsy (PSP); Parkinson Disease (PD) and other Parkinson-like diseases (called Parkinsonisms); Amyotrophic lateral sclerosis (ALS); Chacot-Marie-Tooth Disease; Mucopolysaccharidoses; Adrenoleukodystrophy; Niemann-Pick disease; Krabbe's disease; Pelizaeus-Merzbacher disease; Subacute necrotizing encephalomyelopathy of Leigh; and Progressive encephalopathy, edema, hypsarrhythmia and optic atrophy (PEHO) comprising administering to a patient in need of such treatment a topical, periocular, or intraocular formulation, wherein said formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the topical, periocular, or intraocular formulation comprises alpha-tocotrienol quinone. In other embodiments, the topical, periocular, or intraocular formulation additionally comprises a pharmaceutically acceptable vehicle. In other embodiments, the topical, periocular, or intraocular formulation additionally comprises an ophthalmically acceptable vehicle.

In another embodiment, the invention relates to a method of treating or controlling the ocular symptoms associated with trauma, post-surgical complications, traumatic optic neuropathy (TON), and the damage associated with laser therapy including photodynamic therapy (PDT), with surgical light induced iatrogenic retinopathy, and with corneal transplants and stem cell transplant of eye cells, comprising administering to a patient in need of such treatment a topical, periocular, or intraocular formulation, wherein said formulation comprises an ophthalmically effective amount of one or more agents selected from the group consisting of alpha-tocotrienol quinone, beta-tocotrienol quinone, gamma-tocotrienol quinone, delta-tocotrienol quinone, or mixtures thereof. In some embodiments, the topical, periocular, or intraocular formulation comprises alpha-tocotrienol quinone. In other embodiments, the topical, periocular, or intraocular formulation additionally comprises a pharmaceutically acceptable vehicle. In other embodiments, the topical, periocular, or intraocular formulation additionally comprises an ophthalmically acceptable vehicle.

For all the formulations and methods described above, the composition can be used in its reduced form (hydroquinone form) instead of its quinone form when desired.

### DETAILED DESCRIPTION OF THE INVENTION

The present invention discloses compounds, formulations, methods and kits for use in patients. A patient is a mammal, preferably a human.

The active component of the formulation of the present invention is selected from alpha-tocotrienol quinone, beta-tocotrienol quinone, gamma-tocotrienol quinone, delta-tocotrienol quinone, and mixtures thereof. In one embodiment, the formulation of the present invention comprises alpha-tocotrienol quinone as the active component. In other embodiments, the formulations of the present invention comprise one or more tocotrienol quinones of Formula I or mixtures thereof, in a pharmaceutically acceptable vehicle, and in other embodiments, the formulations of the present invention comprise alpha-tocotrienol quinone in a pharmaceutically acceptable vehicle. In other particular embodiments, the formulations are administered orally. In other embodiments, the formulations of the present invention comprise one or more tocotrienol quinones of Formula I or mixtures thereof, in an ophthalmically acceptable vehicle for topical, periocular, or intraocular administration, and in other embodiments, the formulations of the present invention comprise alpha-tocotrienol quinone in an ophthalmically acceptable vehicle.

The formulations of the present invention comprise tocotrienol quinones which can be produced synthetically from the respective tocotrienol by oxidation with suitable oxidizing agents, as for example ceric ammonium nitrate (CAN). Particularly, the formulations of the present invention comprise alpha-tocotrienol quinone (CAS Reg. No. 1401-66-7) produced by oxidation of alpha-tocotrienol. A preferred process for the production of alpha-tocotrienol has been described in co-owned U.S. provisional application USAN 61/197,585 titled “Process for Enrichment and Isolation of alpha-Tocotrienol from Natural Extracts”.

Syntheses of various members of the tocotrienol family in the d,l- or (RS)-form have been published, see for example Schudel et al., Helv. Chim. Acta (1963) 46, 2517-2526; H. Mayer et al., Helv. Chim. Acta (1967) 50, 1376-11393; H.-J. Kabbe et al., Synthesis (1978), 888-889; M. Kajiwara et al., Heterocycles (1980) 14, 1995-1998; S. Urano et al., Chem. Pharm. Bull. (1983) 31, 4341-4345, Pearce et al., J. Med. Chem. (1992), 35, 3595-3606 and Pearce et al., J. Med. Chem. (1994). 37, 526-541. None of these reported processes lead to the natural form of the tocotrienols, but rather produces racemic mixtures. Syntheses of natural form d-tocotrienols have been published. See for example. J. Scott et al., Helv. Chim. Acta (1976) 59, 290-306, Sato et al. (Japanese Patent 63063674); Sato et al. (Japanese Patent No.JP 01233278) and Couladouros et al. (U.S. Pat. No. 7,038,067).

While synthetic and natural tocopherols are readily available in the market, the natural tocotrienol supply is limited, and generally comprises a mixture of tocotrienols. Crude palm oil which is rich in tocotrienols (800-1500 ppm) offers a potential source of natural tocotrienols. Carotech, Malaysia is able to extract and concentrate tocotrienols from crude palm oil, by a process patented in U.S. Pat. No. 5,157,132. Tocomin®-50 typically comprises about 25.32% mixed tocotrienols (7.00% alpha-tocotrienol, 14.42% gamma-tocotrienol, 3.30% delta-tocotrienol and 0.6% beta-tocotrienol), 6.90% alpha-tocopherol and other phytonutrients such as plant squalene, phytosterols, co-enzyme Q10 and mixed carotenoids.

Other methods for isolation or enrichment of tocotrienol from certain plant oils and plant oil by-products have been described in the literature. For some examples of such isolation and purification processes, see for instance Top A. G. et al., U.S. Pat. No. 5,190,618; Lane R et al., U.S. Pat. No. 6,239,171; Bellafiore, L. et al. U.S. Pat. No. 6,395,915; May, C. Y et al., U.S. Pat. No. 6,656,358; Jacobs, L et al., U.S. Pat. No. 6,838,104; Sumner, C et al. Int. Pat. Pub. WO 99/38860, or Jacobs, L, Int. Pat. Pub. WO 02/500054. The compounds for use in the present invention and the other therapeutically active agents can be administered at the recommended maximum clinical dosage or at lower doses. Dosage levels of the active compounds in the compositions for use in the present invention may be varied so as to obtain a desired therapeutic response depending on the route of administration, severity of the disease and the response of the patient. When administered in combination with other therapeutic agents, the therapeutic agents can be formulated as separate compositions that are given at the same time or different times, or the therapeutic agents can be given as a single composition.

In one embodiment, the purity of the preparation of the compound, such as a tocotrienol quinone preparation, is measured prior to the addition of any pharmaceutical carriers or excipients, or any additional active agents. For example, if alpha-tocotrienol quinone is prepared according to any of the methods described in International Patent Application No. PCT/US2009/062212 or U.S. patent application Ser. No. 12/606,923, the purity of the alpha-tocotrienol quinone is measured on the final product of the method selected, and prior to adding the pharmaceutical carrier(s) or excipient(s) or additional active agent(s). The purity of the desired tocotrienol quinone, or other compound, by weight, can be at least about 20%, at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99%, prior to the addition of any pharmaceutical carriers or excipients, or any additional active agents. These same numerical purity levels can also be used as by mole fraction, or by any other relative measurement (such as weight/volume).

In another embodiment, the purity of the preparation of the compound, such as a tocotrienol quinone preparation, is measured as a fraction of the desired tocotrienol quinone relative to the total amount of tocotrienol quinones and (if present) tocotrienols in the preparation. For example, a composition containing 100 mg of alpha-tocotrienol quinone, 50 mg of beta-tocotrienol quinone, and 50 mg of gamma-tocotrienol quinone would be described as 50% alpha tocotrienol quinone by weight, irrespective of the amounts of other non-tocotrienol or non-tocotrienol quinone compounds present in the preparation. This measurement of purity would be the same whether measured before or after addition of pharmaceutical carriers or excipients, or before or after addition of any non-tocotrienol/non-tocotrienol quinone active agents. The purity of the desired tocotrienol quinone, or other compound, by weight, can be at least about 20%, at least about 30%, at least about 40%, at least about 50%, at least about 60%, at least about 70%, at least about 75%, at least about 80%, at least about 85%, at least about 90%, at least about 95%, at least about 96%, at least about 97%, at least about 98%, or at least about 99%. These same numerical purity levels can also be used as by mole fraction, or by any other relative measurement (such as weight/volume).

The compounds used in the methods of the invention may be administered in any suitable form that will provide sufficient plasma levels of the compounds. The compounds can be administered enterally, orally, parenterally, sublingually, by inhalation (e.g. as mists or sprays), rectally, or topically in unit dosage formulations containing conventional nontoxic pharmaceutically acceptable carriers, excipients, adjuvants, and vehicles as desired. For example, suitable modes of administration include oral, subcutaneous, transdermal, transmucosal, iontophoretic, intravenous, intraarterial, intramuscular, intraperitoneal, intranasal (e.g. via nasal mucosa), subdural, rectal, gastrointestinal, and the like, and directly to a specific or affected organ or tissue. The term parenteral as used herein includes subcutaneous injections, intravenous injection, intraarterial injection, intramuscular injection, intrasternal injection, or infusion techniques. The compounds are mixed with pharmaceutically acceptable carriers, excipients, adjuvants, and vehicles appropriate for the desired route of administration.

Oral administration is advantageous due to its ease of implementation and patient (or caretaker) compliance. In certain embodiments, the active compound and acceptable carrier are administered with a food such as cream cheese, peanut butter, or any other food with at least 25% calories from fat, to encourage uptake and absorption of the lipid-soluble quinones of the invention.

The term “nutraceutical” has been used to refer to any substance that is a food or a part of a food and provides medical or health benefits, including the prevention and treatment of disease. Hence, compositions falling under the label “nutraceutical” may range from isolated nutrients, dietary supplements and specific diets to genetically engineered designer foods, herbal products, and processed foods such as cereals, soups and beverages. In a more technical sense, the term has been used to refer to a product isolated or purified from foods, and generally sold in medicinal forms not usually associated with food and demonstrated to have a physiological benefit or provide protection against chronic disease. Accordingly, the compounds described for use herein can also be administered as nutraceutical or nutritional formulations, with additives such as nutraceutically or nutritionally acceptable excipients, nutraceutically or nutritionally acceptable carriers, and nutraceutically or nutritionally acceptable vehicles. Such formulations are sometimes called medical foods. Suitable nutraceutically acceptable excipients may include liquid solutions such as a solution comprising one or more vegetable-derived oils, such as sesame oil, and/or one or more animal-derived oils, and/or one or more fish-derived oils. The compounds of the present invention can also be mixed with fatty food and administered as a medical food.

The compounds described for use herein can be administered in solid form, in liquid form, in aerosol form, or in the form of tablets, pills, powder mixtures, capsules, granules, injectables, creams, solutions, suppositories, enemas, colonic irrigations, emulsions, dispersions, food premixes, and in other suitable forms. The compounds can also be administered in liposome formulations. The compounds can also be administered as prodrugs, where the prodrug undergoes transformation in the treated subject to a form which is therapeutically effective. Additional methods of administration are known in the art.

Injectable preparations, for example, sterile injectable aqueous or oleaginous suspensions, may be formulated according to methods known in the art using suitable dispersing or wetting agents and suspending agents. The sterile injectable preparation may also be a sterile injectable solution or suspension in a nontoxic parenterally acceptable diluent or solvent, for example, as a solution in propylene glycol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables.

Solid dosage forms for oral administration may include capsules, tablets, pills, powders, and granules. In such solid dosage forms, the active compound may be admixed with at least one inert diluent such as sucrose, lactose, or starch. Such dosage forms may also comprise additional substances other than inert diluents, e.g., lubricating agents such as magnesium stearate. In the case of capsules, tablets, and pills, the dosage forms may also comprise buffering agents. Tablets and pills can additionally be prepared with enteric coatings.

Liquid dosage forms for oral administration may include pharmaceutically acceptable emulsions, solutions, suspensions, syrups, and elixirs containing inert diluents commonly used in the art, such as water. Such compositions may also comprise adjuvants, such as wetting agents, emulsifying and suspending agents, cyclodextrins, and sweetening, flavoring, and perfuming agents. Alternatively, the compound may also be administered in neat form if suitable.

The compounds for use in the present invention can also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a compound for use in the present invention, stabilizers, preservatives, excipients, and the like. The preferred lipids are the phospholipids and phosphatidyl cholines (lecithins), both natural and synthetic. Methods to form liposomes are known in the art. See, for example, Prescott, Ed., Methods in Cell Biology, Volume XIV, Academic Press, New York, N.W., p. 33 et seq (1976).

The topical ophthalmic formulations administered according to the present invention may also include various other ingredients, including but not limited to surfactants, tonicity agents, buffers, preservatives, co-solvents and viscosity building agents.

According to the methods of the present invention, a topical ophthalmic formulation comprising one or more compounds of Formula I or mixtures thereof, preferably alpha-tocotrienol quinone and a ophthalmically acceptable carrier for topical ophthalmic administration or implantation into the conjunctival sac or anterior chamber of the eye, is administered to a patient in need thereof. The formulations are formulated in accordance with methods known in the art for the particular route of administration desired.

The topical ophthalmic formulations administered topically, periocularly, or intraocularly comprise an ophthalmically effective amount of one or more compounds of Formula I or mixtures thereof, preferably alpha-tocotrienol quinone. As used herein, an “ophthalmically effective amount” is one which is sufficient to reduce or eliminate signs or symptoms of the ophthalmic disorders described herein. Generally, for formulations intended to be administered topically to the eye in the form of eye drops or eye ointments, the total amount of the tocotrienol quinone will be 0.001 to 1.0% (w/w). When applied as eye drops, 1-2 drops (approximately 20-45 μl each) of such formulations will be administered from once to several times per day.

One route of administration is topical. The compounds of the present invention can be administered as solutions, suspensions, or emulsions (dispersions) in an ophthalmically acceptable vehicle. An “ophthalmically acceptable” component, as used herein, refers to a component which will not cause any significant ocular damage or ocular discomfort at the intended concentration and over the time of intended use. Solubilizers and stabilizers should be non-reactive. An “ophthalmically acceptable vehicle” refers to any substance or combination of substances which are non-reactive with the compounds and suitable for administration to a patient. Suitable vehicles may be non-aqueous liquid media including the physiologically acceptable oils such as silicone oil, USP mineral oil, white oil, poly(ethylene-glycol), a polyethoxylated castor oil and vegetable oils, for example corn oil, peanut oil, or the like. Other suitable vehicles may be aqueous or oil-in-water solutions suitable for topical application to the patient's eyes. These vehicles may be preferred based on ease of formulation, as well as a patient's ability to easily administer such formulations by means of instilling one to two drops of the solutions in the affected eyes. The formulations may also be suspensions, viscous or semi-viscous gels, or other types of solid or semi-solid formulations. and fat bases, such as natural wax e.g. white bees wax, carnauba wax, wool wax (wool fat), purified lanolin, anhydrous lanolin; petroleum wax e.g. solid paraffin, microcrystalline wax; hydrocarbons e.g. liquid paraffin, white petrolatum, yellow petrolatum; or combinations thereof. The formulations may be applied by use of the hands or an applicator such as a wipe, a contact lens, a dropper or a spray. The compounds and formulations for use in the present invention can be administered using a contact lens-based bioactive agent delivery system, such as those described in U.S. Pat. Appl. Pub. No. 2009/0060981.

The topical ophthalmic formulations administered according to the present invention may also include various other ingredients, including but not limited to surfactants, tonicity agents, buffers, preservatives, co-solvents and viscosity building agents.

Various tonicity agents may be employed to adjust the tonicity of the composition, preferably to that of natural tears for ophthalmic compositions. For example, sodium chloride, potassium chloride, magnesium chloride, calcium chloride, dextrose and/or mannitol may be added to the composition to approximate physiological tonicity. Such an amount of tonicity agent will vary, depending on the particular agent to be added. In general, however, the formulations will have a tonicity agent in an amount sufficient to cause the final composition to have an ophthalmically acceptable osmolality (generally about 200-400 mOsm/kg).

An appropriate buffer system (e.g., sodium phosphate, sodium acetate, sodium citrate, sodium borate or boric acid) may be added to the formulations to prevent pH drift under storage conditions. The particular concentration will vary, depending on the agent employed. Preferably, however, the buffer will be chosen to maintain a target pH within the range of pH 6-7.5.

Topical ophthalmic formulations for the treatment of ophthalmic disorders associated with neurodegenerative diseases and disorders may also comprise aqueous carriers designed to provide immediate, short-term relief of dry eye-type conditions. Such carriers can be formulated as a phospholipid carrier or an artificial tears carrier, or mixtures of both. As used herein, “phospholipid carrier” and “artificial tears carrier” refer to aqueous formulations which: (i) comprise one or more phospholipids (in the case of phospholipid carriers) or other compounds, which lubricate, “wet,” approximate the consistency of endogenous tears, aid in natural tear build-up, or otherwise provide temporary relief of dry eye symptoms and conditions upon ocular administration; (ii) are safe; and (iii) provide the appropriate delivery vehicle for the topical administration of an effective amount of one or more of the specified cytokine inhibitors. Examples or artificial tears compositions useful as artificial tears carriers include, but are not limited to, commercial products, such as Tears Naturale®, Tears Naturale II®., Tears Naturale Free®., and Bion Tears®. (Alcon Laboratories, Inc., Fort Worth, Tex.). Examples of phospholipid carrier formulations include those disclosed in U.S. Pat. Nos. 4,804,539 (Guo et al.), 4,883,658 (Holly), 4,914,088 (Glonek), 5,075,104 (Gressel et al.), 5,278,151 (Korb et al.), 5,294,607 (Glonek et al.), 5,371,108 (Korb et al.), 5,578,586 (Glonek et al.); the foregoing patents are incorporated herein by reference to the extent they disclose phospholipid compositions useful as phospholipid carriers of the present invention.

Other compounds designed to lubricate, “wet,” approximate the consistency of endogenous tears, aid in natural tear build-up, or otherwise provide temporary relief of dry eye symptoms and conditions upon ocular administration the eye are known in the art. Such compounds may enhance the viscosity of the composition, and include, but are not limited to: monomeric polyols, such as, glycerol, propylene glycol, ethylene glycol; polymeric polyols, such as, polyethylene glycol, hydroxypropylmethyl cellulose, carboxy methyl cellulose sodium, hydroxypropyl cellulose; dextrans, such as dextran 70; water soluble proteins, such as gelatin; and vinyl polymers, such as, polyvinyl alcohol, polyvinylpyrrolidone, povidone and carbomers.

Other compounds may also be added to the topical ophthalmic formulations of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers. In general, the phospholipid carrier or artificial tears carrier compositions will exhibit a viscosity of 1 to 400 centipoises.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edetate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

The tocotrienol quinones of Formula I or mixtures thereof, of the present invention may be formulated in solutions or suspensions for intraocular administration. The formulations of the present invention may be administered intraocularly following traumatic events involving the retina and optic nerve head tissues, or prior to or during ophthalmic surgery to prevent damage or injury. Formulations useful for intraocular administration will generally be intraocular injection formulations or surgical irrigating solutions.

The compounds of Formula I or mixtures thereof can also be formulated in an ocular irrigating solution used during ophthalmic surgery to treat retinal or optic nerve head damage resulting from trauma due to injury or prevent damage resulting from the invasive nature of the surgery.

The compounds of Formula I or mixtures thereof can also be administered via periocular administration, and may be formulated in solutions or suspensions for periocular administration. The formulations of the present invention may be administered periocularly following traumatic events involving the retina and optic nerve head tissues, or prior to or during ophthalmic surgery to prevent damage or injury. Formulations useful for periocular administration will generally be periocular injection formulations or surgical irrigating solutions. Periocular administration refers to administration to tissues near the eye, such as administration to the tissues or spaces surrounding the eyeball and within the orbit. Periocular administration can take place by injection, deposit, or any other mode of placement. Periocular routes of administration include, but are not limited to, subconjunctival, suprachoroidal, juxtascleral, posterior juxtascleral, sub-Tenon, posterior sub-Tenon, retrobulbar, peribulbar, or laterobulbar delivery. Raghava et al., Expert Opin. Drug Deliv. 1(1):99-114 (2004); Ghate et al. Investigative Opthalmology and Visual Science, 48 (5): 2230 (2007); Karl G. Csaky, Retina Today, pp. 32-35 (March/April 2007); WO 2009/023877; and EP 1611879 describe various routes of periocular administration.

In general, the doses utilized for the above described purposes will vary, but will be in an effective amount to prevent, reduce or ameliorate retina or optic nerve head neuropathy. As used herein, “ophthalmically effective amount” or ‘therapeutically effective amount” refers to that amount of active agent which prevents, reduces or ameliorates retina or optic nerve head neuropathy. The tocotrienol quinones will generally be contained in the topical, periocular, or intraocular formulations contemplated herein in an amount of from about 0.001 to about 10.0% weight/volume (“% w/v”). Preferred concentrations will range from about 0.1 to about 5.0% w/v. Topical formulations will generally be delivered to the eye one to six times a day, at the discretion of a skilled clinician.

### Co-Administered Agents

The formulations of the present invention may contain additional pharmaceutically active agents or may be dosed concurrently with other pharmaceutical compositions. For example, when treating a mammal for the prevention, reduction, treatment or amelioration of glaucomatous retinopathy, the formulations of the present invention may contain additional “anti-glaucoma” agents or may be dosed concurrently or sequentially with anti-glaucoma agent compositions. Examples of anti-glaucoma agents include: prostaglandins or prostanoids, carbonic anhydrase inhibitors, beta-adrenergic agonists and antagonists, alpha-adrenergic agonists or other anti-glaucoma agents known to those skilled in the art.

While the compounds described herein can be administered as the sole active pharmaceutical agent, they can also be used in combination with one or more other agents used in the treatment or suppression of optic myopathies. Representative agents useful in combination with the compounds described herein for the treatment or suppression of optic myopathies include, but are not limited to, Coenzyme Q, including Coenzyme Q10; idebenone; MitoQ; acetylcarnitine (such as acetyl-L-carnitine or acetyl-DL-carnitine); palmitoylcarnitine (such as palmitoyl-L-carnitine or palmitoyl-DL-carnitine); carnitine (such as L-carnitine or DL-carnitine); quercetine; mangosteen; acai; uridine; N-acetyl cysteine (NAC); polyphenols, such as resveratrol; Vitamin A; Vitamin C; lutein; beta-carotene; lycopene; glutathione; fatty acids, including omega-3 fatty acids such as α-linolenic acid (ALA), eicosapentaenoic acid (EPA), and docosahexaenoic acid (DHA); lipoic acid; and lipoic acid derivatives; Vitamin B complex; Vitamin B1 (thiamine); Vitamin B2 (riboflavin); Vitamin B3 (niacin, nicotinamide, or niacinamide); Vitamin B5 (pantothenic acid); Vitamin B6 (pyridoxine or pyridoxamine); Vitamin B7 (biotin); Vitamin B9 (folic acid, also known as Vitamin B11 or Vitamin M); Vitamin B12 (cobalamins, such as cyanocobalamin); inositol; 4-aminobenzoic acid; folinic acid; Vitamin E; other vitamins; and antioxidant compounds.

### Dosages

The compounds used in the methods of the invention can be administered in various amounts. Examples of daily dosages which can be used are an effective amount within the dosage range of about 0.1 mg/kg to about 300 mg/kg body weight, or within about 0.1 mg/kg to about 100 mg/kg body weight, or within about 0.1 mg/kg to about 80 mg/kg body weight, or within about 0.1 mg/kg to about 50 mg/kg body weight, or within about 0.1 mg/kg to about 30 mg/kg body weight, or within about 0.1 mg/kg to about 10 mg/kg body weight, or within about 1.0 mg/kg to about 80 mg/kg body weight, or within about 1.0 mg/kg to about 50 mg/kg body weight, or within about 1.0 mg/kg to about 30 mg/kg body weight, or within about 1.0 mg/kg to about 10 mg/kg body weight, or within about 10 mg/kg to about 80 mg/kg body weight, or within about 50 mg/kg to about 150 mg/kg body weight, or within about 100 mg/kg to about 200 mg/kg body weight, or within about 150 mg/kg to about 250 mg/kg body weight, or within about 200 mg/kg to about 300 mg/kg body weight, or within about 250 mg/kg to about 300 mg/kg body weight, or about or up to about 1, about or up to about 5, about or up to about 10, about or up to about 15, about or up to about 20, about or up to about 25, about or up to about 30, about or up to about 40, about or up to about 50, about or up to about 60, about or up to about 70, about or up to about 75, about or up to about 80, about or up to about 90, about or up to about 100, about or up to about 125, about or up to about 150, about or up to about 175, about or up to about 200, about or up to about 225, about or up to about 250, about or up to about 275, about or up to about 300, about or up to about 325, about or up to about 350, about or up to about 375, about or up to about 400, about or up to about 425, about or up to about 450, about or up to about 500, about or up to about 550, about or up to about 600, about or up to about 650, about or up to about 700, about or up to about 750, about or up to about 800, about or up to about 850, about or up to about 900, about or up to about 950, or about or up to about 1000 mg total. The compound(s) may be administered in a single daily dose, or the total daily dosage may be administered in divided dosage of two, three or four times daily. These dosages can be administered long term, for example, over months, years, or even over the entire lifetime of the patient.

The particular dosage appropriate for a specific patient is determined by dose titration. For example, animal studies of alpha-tocotrienol quinone administration have shown that in rats, at 10 mg/kg, bioavailability is high (˜90%), Cmax=931 ng/mL, Tmax=3.5 h and t_{1/2}=3.5 h. There is less than dose-proportionality since for an increase in doses of 2.4:6:10:20 there is only an increase in AUCs of 1.5:2.8:4.0:6.7. This lack of dose-proportionality may be due to decreased absorption since there is no change in t_{1/2} over dose range. Alpha-tocotrienol quinone tested in rats was safe when given acutely up to 2000 mg/kg. In fasted dogs, at 10 mg/kg, bioavailability is low (˜16%), Cmax=442 ng/mL, Tmax=2.8 h and t_{1/2}=7.6 h.

The single dose and repeat dose plasma profiles for alpha tocotrienol quinone were simulated using a dose adjusted to achieve a Cmax<10 μM and a Cmin>0.5 μM. Assuming a daily dose and linear kinetics, for a 70 kg adult the total dose would need to be 379 mg (5.41 mg/kg) to achieve a C24 h of 220.5 ng/ml (0.5 μM).

The starting dose can be estimated based on the United States Food and Drug Administration guidelines titled “Estimating the Maximum Safe Starting Dose in Initial Clinical Trials for Therapeutics in Adult Healthy Volunteers” (July 2005) as well as the International Conference on Harmonisation of Technical Requirements for Registration of Pharmaceuticals for Human Use (ICH) guidelines titled “Guidance on Non-clinical Safety Studies for the Conduct of Human Clinical Trials and Marketing Authorization for Pharmaceuticals” (July 2008). Per ICH guidelines, predicted exposures from the starting dose should not exceed 1/50th the NOAEL (No-Adverse-Observed-Effect-Level) in the more sensitive species on a mg/m² basis. Following a single oral dose of alpha-tocotrienol quinone, the NOAEL was established to be 500 mg/kg for the female rat, i.e. 3,000 mg/m². This dosage would be equivalent to 81 mg/kg in an adult human. 1/50th of 81 mg/kg is 1.6 mg/kg, i.e. 110 mg for a 70 kg adult, or 16 mg for a 10 kg child. This dose can be administered once, twice, or three times daily.

### Monitoring Treatment Efficacy

Routine plasma analytes: Blood ketone body ratios including lactate: pyruvate, and beta-hydroxy butyrate:acetoacetate reflect electron balance. Alterations in these ratios can be used to assess systemic metabolic function. Increased blood lactate, increased blood pyruvate, increased blood alanine, and blood pH (to check for metabolic acidosis) can also be monitored.

Metabolomic analysis of plasma and urine: Urine analysis can be performed on the patient, and can include measurement of the following organic acids: lactic acid, pyruvic acid, succinic acid, fumaric acid, 2-ketoglutaric acid, methyl malonic acid, 3-OH butyric acid, acetoacetic acid, 2-keto-3-methylvaleric acid, 2-keto-isocaproic acid, 2-keto-isovaleric acid, ethylmalonic acid, adipic acid, suberic acid, sebacic acid, 4-OH-phenylacetic acid, 4-OH-phenyllacetic acid, 4-OH-phenylpyruvic acid, succinylacetone, and creatinine. Urine analysis performed on the patient can also include measurement of the following amino acids: proline, glutamine, threonine, serine, glutamic acid, arginine, glycine, alanine, histidine, lysine, valine, asparagine, methionine, phenylalanine, isoleucine, leucine, tyrosine, hydroxyproline, creatinine, aspartic acid, cysteine, ornithine, citrulline, homocysteine, and taurine. In a panel of metabolic analytes, the following can be measured: sodium, potassium, chloride, bicarbonate, anion gap, glucose (serum), urea nitrogen (blood), creatinine, calcium, bilirubin, aspartate amino transferase, alanine amino transferase, alkaline phosphatase, total protein (serum), albumin (serum), and hemolysis index. Recently, the Critical Path Initiative has put forth a battery of biomarkers to predict drug toxicity that can also reflect renal mitochondrial function. Alterations in KIM-1, Albumin, Total Protein, β2-microglobulin, Cystatin C, Clusterin, Trefoil Factor-3, and Neutrophil Gelatinase-Associated Lipocalin can be used to both detect (if present) a subclinical nephropathy and assemble a more accurate depiction of the natural history of SURF1 renal function. Finally, Haas, et al. Mol Genet Metab. (2008) 94(1):16-37 describes various tests, such as MRS-based biochemical analysis, that can be used in the present invention.

Optical Coherence Tomography (OCT): OCT is a non-invasive technology used for imaging the retina, the multi-layered sensory tissue lining the back of the eye. OCT, the first instrument to allow doctors to see cross-sectional images of the retina, is revolutionizing the early detection and treatment of eye conditions such as macular holes, pre-retinal membranes, macular swelling and even optic nerve damage.

Retinal thickness may also be measured using other devices such as the Retinal Thickness Analyzer (RTA; Talia Technology, Ltd., Mevasseret Zion, Israel) and the Heidelberg Retina Tomograph (HRT; Heidelberg Engineering GmbH, Heidelberg, Germany). Persons skilled in the art will appreciate that the slope of retinal thickness may be calculated over any number of distances, and that the smallest distance is only limited by the resolution of the devices used to practice the methods of the invention.

Ishihara Color Test: The Ishihara Color test is a test for red-green color deficiencies. The test consists of a number of colored plates, called Ishihara plates, each of which contain a circle of dots appearing randomized in color and size. Within the pattern are dots which form a number visible to those with normal color vision and invisible, or difficult to see, for those with a red-green color vision defect. The full test consists of 38 plates, but the existence of a deficiency is usually clear after a few plates. Testing the first 24 plates gives a more accurate diagnosis of the severity of the color vision defect.

Common plates include a circle of dots in shades of green and light blues with a FIGURE differentiated in shades of brown, or a circle of dots in shades of red, orange and yellow with a FIGURE in shades of green; the first testing for protanopia and the second for deuteranopia.

### Kits

The invention also provides articles of manufacture and kits containing materials useful for treating optic myopathies excluding LHON and excluding DOA. The article of manufacture comprises a container with a label. Suitable containers include, for example, bottles, vials, and test tubes. The containers may be formed from a variety of materials such as glass or plastic. The container holds a compound selected from alpha-tocotrienol quinone, beta-tocotrienol quinone, gamma-tocotrienol quinone, and delta-tocotrienol quinone, or a composition comprising an active agent selected from alpha-tocotrienol quinone, beta-tocotrienol quinone, gamma-tocotrienol quinone, and delta-tocotrienol quinone. In one embodiment, the compound is alpha-tocotrienol quinone. In one embodiment, the active agent is alpha-tocotrienol quinone. The label on the container indicates that the composition is used for treating optic myopathies, and may also indicate directions for use in treatment.

The invention also provides kits comprising any one or more of a compound selected from alpha-tocotrienol quinone, beta-tocotrienol quinone, gamma-tocotrienol quinone, and delta-tocotrienol quinone, or a composition comprising an active agent selected from alpha-tocotrienol quinone, beta-tocotrienol quinone, gamma-tocotrienol quinone, and delta-tocotrienol quinone. In some embodiments, the kit of the invention comprises the container described above, which holds a compound selected from alpha-tocotrienol quinone, beta-tocotrienol quinone, gamma-tocotrienol quinone, and delta-tocotrienol quinone, or a composition comprising an active agent selected from alpha-tocotrienol quinone, beta-tocotrienol quinone, gamma-tocotrienol quinone, and delta-tocotrienol quinone. In other embodiments, the kit of the invention comprises the container described above, which holds a compound selected from alpha-tocotrienol quinone, beta-tocotrienol quinone, gamma-tocotrienol quinone, and delta-tocotrienol quinone, or a composition comprising an active agent selected from alpha-tocotrienol quinone, beta-tocotrienol quinone, gamma-tocotrienol quinone, and delta-tocotrienol quinone, and a second container comprising a vehicle for the compound or composition, such as one or more vegetable-derived oils, such as sesame oil, and/or one or more animal-derived oils, and/or one or more fish-derived oils. In other embodiments, the kit of the invention comprises the container described above, which holds a compound selected from alpha-tocotrienol quinone, beta-tocotrienol quinone, gamma-tocotrienol quinone, and delta-tocotrienol quinone, or a composition comprising an active agent selected from alpha-tocotrienol quinone, beta-tocotrienol quinone, gamma-tocotrienol quinone, and delta-tocotrienol quinone, where the compound or composition has been pre-mixed with a vehicle for the compound or composition, such as one or more vegetable-derived oils, such as sesame oil, and/or one or more animal-derived oils, and/or one or more fish-derived oils. The kits may further include other materials desirable from a commercial and user standpoint, including other vehicles, buffers, diluents, filters, needles, syringes, and package inserts with instructions for performing any of the methods described herein for treatment of optic myopathies excluding LHON and excluding DOA.

In other aspects, the kits may be used for any of the methods described herein, including, for example, to treat an individual with optic myopathies excluding LHON and excluding DOA.

### EXAMPLES

### Example 1

### FRDA Cell Line Assay and Initial Screen for Effective Compounds

Alpha-Tocotrienol quinone was tested for its ability to rescue Friedreich's Ataxia (FRDA) fibroblast cells obtained from the Coriell Cell Repositories (Camden, N.J.; repository number GM04078), from stress effected by addition of L-buthionine-(S,R)-sulfoximine (BSO), as described in Jauslin et al., Hum. Mol. Genet. 11(24):3055 (2002), Jauslin et al., FASEB J. 17:1972-4 (2003), and International Patent Application WO 2004/003565. EC50 concentrations of test compound and its redox-silent version were determined and compared.

MEM (a medium enriched in amino acids and vitamins, catalog no. 1-31F24-I) and Medium 199 (M199, catalog no. 1-21F22-I) with Earle's Balanced Salts, without phenol red, were purchased from Bioconcept. Fetal Calf Serum was obtained from PAA Laboratories. Basic fibroblast growth factor and epidermal growth factor were purchased from PeproTech. Penicillin-streptomycin-glutamine mix, L-buthionine (S,R)-sulfoximine, and insulin from bovine pancreas were purchased from Sigma. Calcein AM was purchased from Molecular Probes. Cell culture medium was made by combining 125 mL M199 EBS, 50 ml Fetal Calf Serum, 100 U/mL penicillin, 100 μg/ml streptomycin, 2 mM glutamine, 10 μg/mL insulin, 10 ng/mL EGF, and 10 ng/mL bFGF. MEM EBS was added to make the volume up to 500 mL. A 10 mM BSO solution was prepared by dissolving 444 mg BSO in 200 mL of medium with subsequent filter-sterilization. During the course of the experiments, this solution was stored at +4° C.

The test samples were supplied in 1.5 mL glass vials. The compounds were diluted with DMSO, ethanol or PBS to result in a 5 mM stock solution. Once dissolved, they were stored at −20° C.

Test samples were screened according to the following protocol: A culture with FRDA fibroblasts was started from a 1 mL vial with approximately 500,000 cells stored in liquid nitrogen. Cells were propagated in 10 cm cell culture dishes by splitting every third day in a ratio of 1:3 until nine plates were available. Once confluent, fibroblasts were harvested. For 54 micro titer plates (96 well-MTP) a total of 14.3 million cells (passage eight) were re-suspended in 480 mL medium, corresponding to 100 μL medium with 3,000 cells/well. The remaining cells were distributed in 10 cm cell culture plates (500,000 cells/plate) for propagation. The plates were incubated overnight at 37° C. in an atmosphere with 95% humidity and 5% CO₂ to allow attachment of the cells to the culture plate.

MTP medium (243 μL) was added to a well of the microtiter plate. The test compounds were unfrozen, and 7.5 μL of a 5 mM stock solution was dissolved in the well containing 243 μL medium, resulting in a 150 μM master solution. Serial dilutions from the master solution were made. The period between the single dilution steps was kept as short as possible (generally less than 1 second).

Plates were kept overnight in the cell culture incubator. The next day, 10 μL of a 10 mM BSO solution were added to the wells, resulting in a 1 mM final BSO concentration. Forty-eight hours later, three plates were examined under a phase-contrast microscope to verify that the cells in the 0% control (wells E1-H1) were clearly dead. The medium from all plates was discarded, and the remaining liquid was removed by gently tapping the plate inversed onto a paper towel.

100 μL of PBS containing 1.2 μM Calcein AM were then added to each well. The plates were incubated for 50-70 minutes at room temperature. After that time the PBS was discarded, the plate gently tapped on a paper towel and fluorescence (excitation/emission wavelengths of 485 nm and 525 nm, respectively) was read on a Gemini fluorescence reader. Data was imported into Microsoft Excel (EXCEL is a registered trademark of Microsoft Corporation for a spreadsheet program) and used to calculate the EC₅₀ concentration for each compound.

The compounds were tested three times, i.e., the experiment was performed three times, the passage number of the cells increasing by one with every repetition.

The solvents (DMSO, ethanol, PBS) neither had a detrimental effect on the viability of non-BSO treated cells nor did they have a beneficial influence on BSO-treated fibroblasts even at the highest concentration tested (1%). The compounds showed no auto-fluorescence. The viability of non-BSO treated fibroblasts was set as 100%, and the viability of the BSO- and compound-treated cells was calculated as relative to this value.

Alpha-tocotrienol quinone protects the FRDA cells with an ED₅₀ of 37 nM.

### Example 2

### Treatment of a Female Diagnosed with Friedreich's Ataxia

A female patient with Friedreich's Ataxia is treated with alpha-tocotrienol quinone. Alpha-tocotrienol quinone is administered to the patient orally; the drug is mixed with sesame oil for administration, and the intake is taken with a fatty food such as yoghurt or ice cream. The following dosing of alpha-tocotrienol quinone is used:

On Day 1 the dose is 100 mg TID. It is escalated on Day 8 to 200 mg TID and continued at this dosage.

While being treated with alpha tocotrienol quinone, the patient's medical team monitors the patient's eyes for any signs of improvement or signs of worsening of the disease by measuring visual acuity, color vision, vision field and OCT.

Close monitoring of the patient during the study is performed, to detect any adverse events. In addition, the investigator is authorized to stop the study if the safety of the subject is at risk.

The disclosures of all publications, patents, patent applications and published patent applications referred to herein by an identifying citation are hereby incorporated herein by reference in their entirety.

Although the foregoing invention has been described in some detail by way of illustration and example for purposes of clarity of understanding, it is apparent to those skilled in the art that certain minor changes and modifications will be practiced. Therefore, the description and examples should not be construed as limiting the scope of the invention.

What is claimed is:

1. A formulation for preventing, reducing, ameliorating or treating ophthalmic disorders, or for stopping the progression of, or reversing, the loss of vision in a patient, wherein the formulation comprises an ophthalmically effective amount of one or more agents selected from the group consisting of alpha-tocotrienol quinone, beta-tocotrienol quinone, gamma-tocotrienol quinone, delta-tocotrienol quinone, the corresponding hydroquinones thereof, or mixtures thereof.

2. The formulation according to claim 1, wherein the therapeutically effective ophthalmic agent is alpha-tocotrienol quinone.

3. The formulation according to claim 1, additionally comprising a pharmaceutically acceptable vehicle.

4. The formulation according to claim 1, additionally comprising an ophthalmically acceptable vehicle.

5. The formulation according to claim 2, wherein the alpha-tocotrienol quinone has a purity of about 75% to about 99%.

6. A method of treating or controlling the ocular symptoms associated with neurodegenerative diseases or trauma, comprising administering to a patient in need thereof a formulation comprising an ophthalmically effective amount of one or more agents selected from the group consisting of alpha-tocotrienol quinone, beta-tocotrienol quinone, gamma-tocotrienol quinone, delta-tocotrienol quinone, the corresponding hydroquinones thereof, or mixtures thereof.

7. The method of claim 6, wherein the formulation is administered orally.

8. The method of claim 6, wherein the formulation is administered topically in eye drops.

9. The method of claim 6, wherein the ophthalmic formulation is administered topically in an irrigating solution.

10. The method of claim 6, wherein the formulation is administered periocularly.

11. The method of claim 6, wherein the formulation is administered intraocularly.

12. The method according to claim 6, wherein the formulation is an oral formulation comprising an ophthalmically effective amount of alpha-tocotrienol quinone.

13. The method according to claim 12, wherein the oral formulation additionally comprises a pharmaceutically acceptable vehicle.

14. The method according to claim 6, wherein the ocular symptoms are associated with inherited mitochondrial diseases; Chronic Progressive External Opthalmoplegia (CPEO); Spinocerebellar ataxia (SCA), also called Machado-Joseph disease; Leigh's Syndrome; Friedreich's ataxia (FRDA); Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency.

15. The method according to claim 14, wherein the ocular symptoms are associated with Friedreich's ataxia (FRDA); Mitochondrial Myopathy, Encephalopathy, Lactacidosis, and Stroke (MELAS); Myoclonic Epilepsy with Ragged Red Fibers (MERRF); Leigh's syndrome; Kearns-Sayre Syndrome (KSS); overlap syndromes; or Chronic Progressive External Opthalmoplegia (CPEO).

16. The method according to claim 6, wherein the ocular symptoms are associated with neurodegenerative diseases; Parkinson's disease; Alzheimer's disease; Amyotrophic Lateral Sclerosis (ALS); motor neuron diseases; Huntington's Disease; age-associated diseases; glaucoma; disorders of the outer retina, macular degeneration, age related macular degeneration and juvenile macular degeneration.

17. The method according to claim 6, wherein the ocular symptoms are associated with diabetic retinopathy; Progressive Supranuclear Palsy (PSP); Parkinson-like diseases; Chacot-Marie-Tooth Disease; Mucopolysaccharidoses; Adrenoleukodystrophy; Niemann-Pick disease; Krabbe's disease; Pelizaeus-Merzbacher disease; Subacute necrotizing encephalomyelopathy of Leigh; and Progressive Encephalopathy, Edema, Hypsarrhythmia and Optic Atrophy (PEHO).

18. The method according to claim 6, wherein the ocular symptoms are associated with trauma.

19. The method according to claim 18, wherein the ocular symptoms are selected from retinal ischemia, acute retinopathies associated with trauma, post-surgical complications, the damage associated with laser therapy including photodynamic therapy (PDT), traumatic optic neuropathy (TON), the damage associated with surgical light induced iatrogenic retinopathy, the damage associated with corneal transplants, and the damage associated with stem cell transplant of eye cells.

20. The method according to claim 12, wherein the ocular symptoms are associated with mitochondrial diseases selected from: Chronic Progressive External Opthalmoplegia (CPEO); Spinocerebellar ataxia (SCA), also called Machado-Joseph disease; Leigh's Syndrome; Friedreich's ataxia (FRDA); Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency.

21. The method according to claim 20, wherein the ocular symptoms are associated with Friedreich's ataxia (FRDA); Mitochondrial Myopathy, Encephalopathy, Lactacidosis, and Stroke (MELAS); Myoclonic Epilepsy with Ragged Red Fibers (MERRF); Leigh's syndrome; Kearns-Sayre Syndrome (KSS); overlap syndromes; or Chronic Progressive External Opthalmoplegia (CPEO).

22. The method according to claim 12, wherein the ocular symptoms are associated with neurodegenerative diseases selected from Parkinson's disease; Alzheimer's disease; Amyotrophic Lateral Sclerosis (ALS); motor neuron diseases; Huntington's Disease; age-associated diseases; glaucoma; disorders of the outer retina, macular degeneration; age related macular degeneration and juvenile macular degeneration.

23. The method according to claim 12, wherein the ocular symptoms are associated with trauma.

24. The method according to claim 23, wherein the ocular symptoms are selected from retinal ischemia, acute retinopathies associated with trauma, post-surgical complications, traumatic optic neuropathy (TON), and the damage associated with laser therapy including photodynamic therapy (PDT), the damage associated with surgical light induced iatrogenic retinopathy, the damage associated with corneal transplants, and the damage associated with stem cell transplant of eye cells.

25. A method of treating or controlling the ocular symptoms associated with neurodegenerative diseases or trauma, comprising administering to a patient in need thereof a topical ophthalmic formulation comprising an ophthalmically effective amount of one or more agents selected from the group consisting of alpha-tocotrienol quinone, beta-tocotrienol quinone, gamma-tocotrienol quinone, delta-tocotrienol quinone, the corresponding hydroquinones thereof, or mixtures thereof.

26. The method according to claim 25, wherein the topical ophthalmic formulation comprises an ophthalmically effective amount of alpha-tocotrienol quinone.

27. The method according to claim 26, wherein the topical ophthalmic formulation additionally comprises an ophthalmically acceptable vehicle.

28. The method according to claim 25, wherein the ocular symptoms are associated with mitochondrial diseases selected from; Chronic Progressive External Opthalmoplegia (CPEO); Spinocerebellar ataxia (SCA), also called Machado-Joseph disease; Leigh's Syndrome; Friedreich's ataxia (FRDA); Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and
Complex V deficiency.

29. The method according to claim 28, wherein the ocular symptoms are associated with Friedreich's ataxia (FRDA); Mitochondrial Myopathy, Encephalopathy, Lactacidosis, and Stroke (MELAS); Myoclonic Epilepsy with Ragged Red Fibers (MERRF); Leigh's syndrome; Kearns-Sayre Syndrome (KSS); overlap syndromes; or Chronic Progressive External Opthalmoplegia (CPEO).

30. The method according to claim 25, wherein the ocular symptoms are associated with neurodegenerative diseases selected from Parkinson's disease; Alzheimer's disease; Amyotrophic Lateral Sclerosis (ALS); motor neuron diseases; Huntington's Disease; age-associated diseases; glaucoma; disorders of the outer retina, macular degeneration, age related macular degeneration and juvenile macular degeneration.

31. The method according to claim 25, wherein the ocular symptoms are associated with trauma.

32. The method according to claim 31, wherein the ocular symptoms are selected from retinal ischemia, acute retinopathies associated with trauma, post-surgical complications, traumatic optic neuropathy (TON), and the damage associated with laser therapy including photodynamic therapy (PDT), the damage associated with surgical light induced iatrogenic retinopathy, the damage associated with corneal transplants, and the damage associated with stem cell transplant of eye cells.

33. The method according to claim 26, wherein the ocular symptoms are associated with mitochondrial diseases selected from Chronic Progressive External Opthalmoplegia (CPEO); Spinocerebellar ataxia (SCA), also called Machado-Joseph disease; Leigh's Syndrome; Friedreich's ataxia (FRDA); Mitochondrial Myopathy, Encephalopathy, Lactacidosis, and Stroke (MELAS); Myoclonic Epilepsy with Ragged Red Fibers (MERRF); Kearns-Sayre Syndrome (KSS); overlap syndromes; Co-Enzyme Q10 (CoQ10) Deficiency; Complex I deficiency; Complex II deficiency; Complex III deficiency; Complex IV deficiency; and Complex V deficiency.

34. The method according to claim 33, wherein the ocular symptoms are associated with Friedreich's ataxia (FRDA); Mitochondrial Myopathy, Encephalopathy, Lactacidosis, and Stroke (MELAS); Myoclonic Epilepsy with Ragged Red Fibers (MERRF); Leigh's syndrome; Kearns-Sayre Syndrome (KSS); overlap syndromes; or Chronic Progressive External Opthalmoplegia (CPEO).

35. The method according to claim 26, wherein the ocular symptoms are associated with neurodegenerative diseases selected from Parkinson's disease; Alzheimer's disease; Amyotrophic Lateral Sclerosis (ALS); motor neuron diseases; Huntington's Disease; age-associated diseases; glaucoma; disorders of the outer retina, macular degeneration; age related macular degeneration and juvenile macular degeneration.

36. The method according to claim 27, wherein the ocular symptoms are associated with trauma.

37. The method according to claim 36, wherein the ocular symptoms are selected from retinal ischemia, acute retinopathies associated with trauma, post-surgical complications, traumatic optic neuropathy (TON), and the damage associated with laser therapy including photodynamic therapy (PDT), the damage associated with surgical light induced iatrogenic retinopathy, the damage associated with corneal transplants, and the damage associated with stem cell transplant of eye cells.
